(19) United States
(12) Patent Application Publication
(10) Pub. No.: US 2021/0189243 A1
(43) Pub. Date: Jun. 24, 2021

(54) COMPOUND, MIXTURE, LIQUID CRYSTAL COMPOSITION, CURED PRODUCT, OPTICALLY ANISOTROPIC BODY, AND REFLECTIVE FILM

(71) Applicant: FUJIFILM Corporation, Tokyo (JP)

(72) Inventors: Keisuke KODAMA, Minami-ashigara-shi (JP); Yuko SUZUKI, Minami-ashigara-shi (JP); Shunya KATOH, Minami-ashigara-shi (JP)

(73) Assignee: FUJIFILM Corporation, Tokyo (JP)

(21) Appl. No.: 17/193,392

(22) Filed: Mar. 5, 2021

Related U.S. Application Data
(63) Continuation of application No. PCT/JP2019/034809, filed on Sep. 4, 2019.

(30) Foreign Application Priority Data
Sep. 7, 2018 (JP) 2018-168305

(51) Int. Cl. C09K 19/58; C09K 19/38; C07D 321/10; C07F 7/08; G02B 5/26

(52) U.S. Cl. CPC C09K 19/588; C09K 19/3852; C09K 2019/0448; C07F 7/0812; G02B 5/26; C07D 321/10

(57) ABSTRACT

An object of the present invention is to provide a compound which increases the intensity of HTP by exposure to irradiation with light such as ultraviolet rays, and a mixture including the compound. Another object of the present invention is to provide a liquid crystal composition, a cured product, an optically anisotropic body, and a reflective film.

The compound of the present invention is represented by General Formula (1).

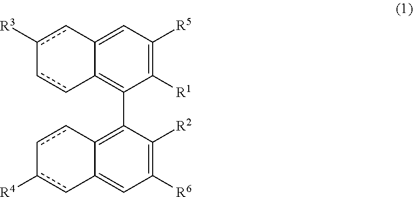

### CROSS-REFERENCE TO RELATED APPLICATIONS

This application is a Continuation of PCT International Application No. PCT/JP2019/034809 filed on Sep. 4, 2019, which claims priority under 35 U.S.C. § 119(a) to Japanese Patent Application No. 2018-168305 filed on Sep. 7, 2018. The above application is hereby expressly incorporated by reference, in its entirety, into the present application.

### BACKGROUND OF THE INVENTION

### 1. Field of the Invention

The present invention relates to a compound, a mixture, a liquid crystal composition, a cured product, an optically anisotropic body, and a reflective film.

### 2. Description of the Related Art

A compound exhibiting liquid crystallinity (hereinafter, also referred to as a “liquid crystalline compound”) can be applied to various uses. For example, the liquid crystalline compound is applied to the manufacturing of an optically anisotropic body typified by a retardation film, or to the manufacturing of a reflective film obtained by immobilizing a cholesteric liquid crystalline phase.

Generally, the cholesteric liquid crystalline phase is formed by adding a chiral compound to a nematic liquid crystal. Among these, as a chiral compound having a strong helical twisting power (HTP), a binaphthyl derivative is usually used.

JP2003-055315A discloses, as an intermediate of the binaphthyl derivative, a binaphthol derivative including a stilbene structure in a molecule. In the synthesis method disclosed in the section of Example of JP2003-055315A, a binaphthol derivative including only a trans-stilbene structure as the stilbene structure is selectively synthesized.

### SUMMARY OF THE INVENTION

On the other hand, in recent years, a chiral compound which increases the intensity of HTP by exposure to irradiation with light such as ultraviolet rays has been desired. As a result of studies on the binaphthol derivative disclosed in JP2003-055315A, the present inventors have found that the intensity of HTP is reduced by exposure, which does not satisfy the desired requirements.

Therefore, an object of the present invention is to provide a compound which increases the intensity of HTP by exposure to irradiation with light such as ultraviolet rays, and a mixture including the compound.

Another object of the present invention is to provide a liquid crystal composition, a cured product, an optically anisotropic body, and a reflective film.

The present inventors have found that the above-described objects can be achieved by a compound represented by General Formula (1) described later, and have completed the present invention.

That is, the present inventors have found that the above-described object can be achieved by the following configuration.

[1] A compound represented by General Formula (1) described later.

[2] The compound according to [1],

in which L¹ represents a single bond.

[3] The compound according to [1] or [2],

in which one or more groups selected from the group consisting of R¹, R³, and R⁵ represent the monovalent substituent represented by General Formula (2), and one or more groups selected from the group consisting of R², R⁴, and R⁶ represent the monovalent substituent represented by General Formula (2).

[4] The compound according to any one of [1] to [3],

in which R¹ and R² are bonded to each other to form a ring structure.

[5] The compound according to any one of [1] to [4],

in which L¹ represents the divalent linking group represented by General Formula (3),

Rⁱ represents *-L^{S1}-aromatic hydrocarbon ring group, or

R¹ and R² are bonded to each other to represent *-L^{S2}-divalent aromatic hydrocarbon ring group-L^{S2}-*,

where, L^{S1} and L^{S2} each independently represent a single bond or a divalent linking group, and * represents a bonding position.

[6] The compound according to any one of [1] to [5],

in which R⁷ and R⁸ represent hydrogen atoms.

[7] The compound according to any one of [1] to [6],

in which Ar¹ represents a benzene ring group.

[8] The compound according to any one of [1] to [7],

in which all of R¹ to R⁶ represent a monovalent substituent other than a monovalent substituent represented by General Formula (4) described later.

[9] A mixture consisting of the compound according to [8] and a compound represented by General Formula (Y1) described later.

[10] The mixture according to [9],

in which a ratio of a content of the monovalent substituent represented by General Formula (2) to a content of the monovalent substituent represented by General Formula (6) is 5 or more.

[11] A liquid crystal composition comprising:

a liquid crystalline compound; and

the compound according to any one of [1] to [8] or the mixture according to [9] or [10].

[12] A cured product obtained by curing the liquid crystal composition according to [11].

[13] An optically anisotropic body formed by using the liquid crystal composition according to [11].

[14] A reflective film formed by using the liquid crystal composition according to [11].

According to the present invention, it is possible to provide a compound which increases the intensity of HTP by exposure to irradiation with light such as ultraviolet rays, and a mixture including the compound.

In addition, according to the present invention, it is also possible to provide a liquid crystal composition, a cured product, an optically anisotropic body, and a reflective film.

### DESCRIPTION OF THE PREFERRED EMBODIMENTS

Hereinafter, the present invention will be described in detail.

The description of the constitutional requirements described below is made on the basis of representative embodiments of the present invention, but it should not be construed that the present invention is limited to those embodiments.

In the present specification, a numerical range represented using “to” means a range including numerical values described before and after the preposition “to” as a lower limit value and an upper limit value.

In addition, in the present specification, “(meth)acryloyl” means acryloyl or methacryloyl.

[Compound Represented by General Formula (1)]

As a feature of a compound (hereinafter, also referred to as a “specific compound”) represented by General Formula (1), at least one of R′, R², R³, R⁴, R⁵, or R⁶ includes a monovalent substituent represented by General Formula (2) described later. In other words, by including a monovalent substituent represented by General Formula (2) described later, the specific compound has a cis-stilbene derivative structure introduced into the molecule. The monovalent substituent represented by General Formula (2) described later photoisomerizes to a trans-stilbene derivative structure in a case of being irradiated with energy such as ultraviolet rays, and as a result, the intensity of HTP of the specific compound increases.

In addition, as described later, in a case where R¹ and R² in the specific compound are bonded to each other to form a ring structure, since the rotation of a binaphthyl skeleton in the specific compound is suppressed, the temperature dependence of HTP is low (in other words, HTP is unlikely to change depending on the temperature), and furthermore, HTP after exposure is high.

In the present specification, “binaphthyl skeleton” means a structural site (structural site shown below) of General Formula (1) described later, excluding R¹ to R⁶. That is, the structural site generically corresponds to structural sites of General Formula (1-1) and General Formula (1-2) described later, excluding R′ to R⁶.

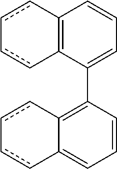

Hereinafter, the specific compound will be described in detail.

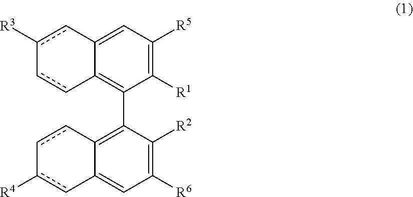

In General Formula (1), R¹ to R⁶ each independently represent a hydrogen atom or a monovalent substituent. However, at least one of R¹, R², R³, R⁴, R⁵, or R⁶ represents a monovalent substituent represented by General Formula (2) described later.

Examples of the monovalent substituent represented by R′ to R⁶ include monovalent substituents such as an alkyl group, an alkoxy group, an aryl group, an arylcarbonyloxy group, an aryloxycarbonyl group, an arylamide group, an alkoxycarbonyl group, an alkylcarbonyloxy group, and a cinnamoyloxy group; a monovalent substituent represented by General Formula (2) described later; and a monovalent substituent represented by General Formula (4) described later.

The alkyl group and an alkyl group in the alkylcarbonyloxy group, represented by R¹ to R⁶, may be linear, branched, or cyclic, and examples thereof include an alkyl group having 1 to 10 carbon atoms (preferably, 1 to 6 carbon atoms).

The alkoxy group and an alkoxy group in the alkoxycarbonyl group, represented by R′ to R⁶, may be linear, branched, or cyclic, and examples thereof include an alkoxy group having 1 to 10 carbon atoms (preferably, 1 to 6 carbon atoms).

Examples of the aryl group, and an aryl group in the arylcarbonyloxy group, aryloxycarbonyl group, and arylamide group, represented by R′ to R⁶, include an aryl group having 6 to 18 carbon atoms (for example, a phenyl group).

The above-described monovalent substituents such as an alkyl group, an alkoxy group, an aryl group, an arylcarbonyloxy group, an aryloxycarbonyl group, an arylamide group, an alkoxycarbonyl group, an alkylcarbonyloxy group, and a cinnamoyloxy group may further have a substituent.

The substituent is not particularly limited, and examples thereof include an alkoxy group having 1 to 10 carbon atoms, a phenoxy group, and a group including a polymerizable group shown below.

Examples of the polymerizable group in the group including a polymerizable group include a known polymerizable group, but from the viewpoint of reactivity, the polymerizable group is preferably a functional group capable of an addition polymerization reaction and more preferably a polymerizable ethylenically unsaturated group or a ring-opening polymerizable group. Examples of the polymerizable group include a (meth)acryloyloxy group, a (meth)acryloylamino group, a vinyl group, a maleimide group, an acetyl group, a styryl group, an allyl group, an epoxy group, an oxetane group, and a group including these groups. A hydrogen atom in each of these groups may be replaced with another substituent such as a halogen atom.

Suitable specific examples of the polymerizable group include groups represented by General Formulae (P-1) to (P-21). In the following formulae, * represents a bonding position. In addition, Ra represents a hydrogen atom or a methyl group. In addition, Me represents a methyl group, and Et represents an ethyl group.

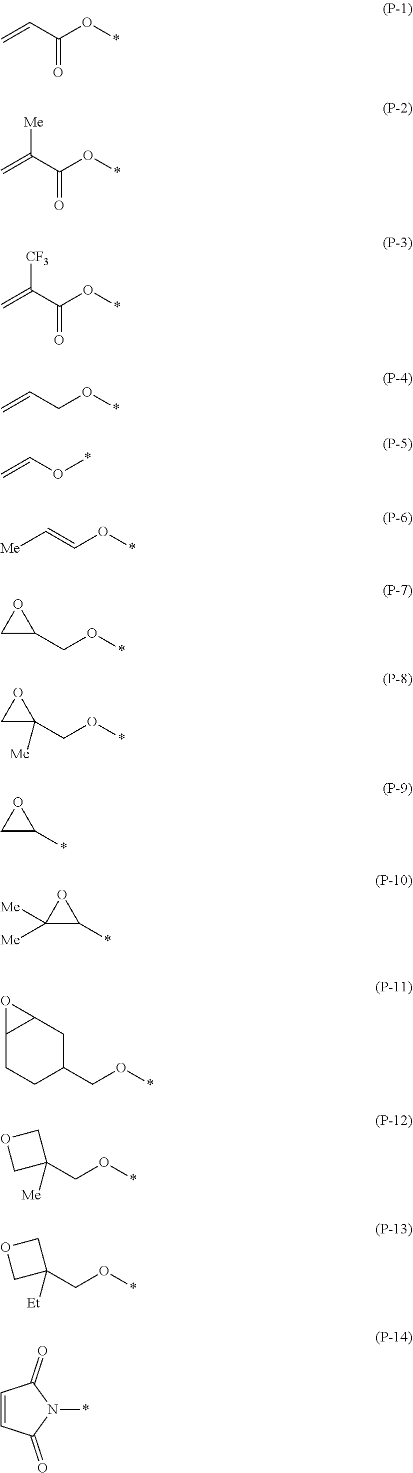

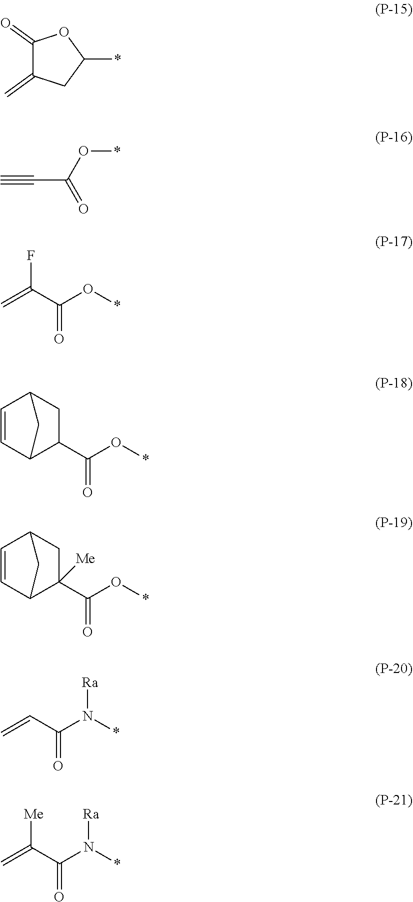

The group including the above-described polymerizable group is not particularly limited as long as it has the above-described polymerizable group, and examples thereof include a group represented by General Formula (PA).

*-L^{A}-P (PA)

In General Formula (PA), L^{A} represents a single bond or a divalent linking group. P represents the group represented by General Formulae (P-1) to (P-21) described above. * represents a bonding position.

As the divalent linking group represented by L^{A}, for example, a linear or branched alkylene group having 1 to 10 carbon atoms, or a divalent linking group of a linear or branched alkylene group having 1 to 10 carbon atoms, in which one or more —CH₂— are replaced with one or more groups selected from the group consisting of —O—, —S—, —NH—, —N(CH₃)—, —CO—, —COO—, and —COO— is preferable.

As the divalent linking group represented by L^{A}, a group of a linear or branched alkylene group having 1 to 10 carbon atoms, in which one or more —CH₂— are replaced with —O— is more preferable.

As the group represented by General Formula (PA), a group represented by “*—O—(CH₂)ₖ—P (k represents an integer of 1 to 10)” is preferable.

For example, in a case where the arylcarbonyloxy group, the aryloxycarbonyl group, or the arylamide group represented by R¹ to R⁶ has a substituent, examples of an arylcarbonyloxy group having a substituent, an aryloxycarbonyl group having a substituent, and an arylamide group having a substituent include a group represented by General Formula (T1).

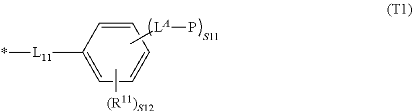

In General Formula (T1), L₁₁ represents —O—CO—, —COO—O—, —N(R_{b})—CO—, or —CO—N(R_{b})—. R_{b} represents a hydrogen atom or an alkyl group having 1 to 10 carbon atoms.

L^{A} and P respectively have the same meanings as L^{A} and P in General Formula (PA) described above, and the suitable aspects are also the same.

R¹¹ represents a monovalent substituent. Examples of the monovalent substituent represented by R¹¹ include an alkoxy group having 1 to 3 carbon atoms.

S11 and S12 each independently represent an integer of 0 to 5. However, 1≤S11+S12≤5.

Among these, as the monovalent substituent represented by R′ to R⁶, an alkoxy group, an arylcarbonyloxy group, an aryloxycarbonyl group, an arylamide group, or the monovalent substituent represented by General Formula (2) described above is preferable. In addition, as described later, R¹ and R² may be bonded to each other to form a ring structure.

Hereinafter, the monovalent substituent represented by General Formula (2) and the monovalent substituent represented by General Formula (4) will be described.

First, the monovalent substituent represented by General Formula (2) will be described.

By including a monovalent substituent represented by General Formula (2), the specific compound has a cis-stilbene derivative structure introduced into the molecule. Specifically, in a case where L¹ in the monovalent substituent represented by General Formula (2) represents a single bond, in an olefin double bonding site (C^{A}═C) specified in General Formula (2), Ar¹ specified in General Formula (2) and the benzene ring (meaning a benzene ring to which the monovalent substituent represented by General Formula (2) is bonded) included in the binaphthyl skeleton of General Formula (1) have a cis-type arrangement to form a cis-stilbene derivative structure. In addition, in a case where L′ in the monovalent substituent represented by General Formula (2) represents a divalent linking group represented by General Formula (3), in the olefin double bonding site (C^{A}═C) specified in General Formula (2), Ar¹ specified in General Formula (2) and Ar² specified in General Formula (3) have a cis-type arrangement to form a cis-stilbene derivative structure.

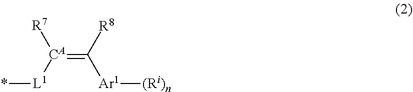

In General Formula (2), Ar¹ represents an (n+1)-valent aromatic hydrocarbon ring group.

An aromatic hydrocarbon ring constituting the aromatic hydrocarbon ring group is not particularly limited, and examples thereof include an aromatic hydrocarbon ring having 6 to 20 carbon atoms. Among these, an aromatic hydrocarbon ring having 6 to 10 carbon atoms is preferable, and a benzene ring is more preferable (that is, Ar′ is more preferably a benzene ring group).

C^{A} represents a carbon atom.

R⁷ and R⁸ each independently represent a hydrogen atom, a cyano group, a substituted boryl group, a substituted silyl group, a substituted aluminum group, a halogen atom, an alkoxycarbonyl group, an alkylcarbonyl group, or a monovalent aliphatic hydrocarbon group having 1 to 10 carbon atoms.

Examples of the substituted boryl group represented by R⁷ and R⁸ include a group represented by *—BR^{X1}R^{X2} (R^{X1} and R^{X2} each independently represent a hydrogen atom or a monovalent substituent, in which at least one of R^{X1} or R^{X2} represents a monovalent substituent; R^{X1} and R^{X2} may be bonded to each other to form a ring structure).

The monovalent substituent represented by R^{X1} and R^{X2} is not particularly limited, and examples thereof include an alkyl group having 1 to 10 carbon atoms (which may be linear, branched, or cyclic), a phenyl group, and an alkoxy group having 1 to 10 carbon atoms.

Examples of the substituted silyl group represented by R⁷ and R⁸ include a group represented by *—SiR^{X3}R^{X4}R^{X5} (R^{X3} to R^{X5} each independently represent a monovalent substituent).

The monovalent substituent represented by R^{X3} to R^{X5} is not particularly limited, and examples thereof include an alkyl group having 1 to 10 carbon atoms (which may be linear, branched, or cyclic) and a phenyl group.

Examples of the substituted aluminum group represented by R⁷ and R⁸ include a group represented by *—AlR^{X5}R^{X6} (R^{X5} and R^{X6} each independently represent a hydrogen atom or a monovalent substituent, in which at least one of R^{X5} or R^{X6} represents a monovalent substituent; R^{X5} and R^{X6} may be bonded to each other to form a ring structure).

The monovalent substituent represented by R^{X5} and R^{X6} is not particularly limited, and examples thereof include an alkyl group having 1 to 10 carbon atoms (which may be linear, branched, or cyclic) and a phenyl group.

Examples of the halogen atom represented by R⁷ and R⁸ include a fluorine atom, a chlorine atom, a bromine atom, and an iodine atom.

An alkyl group in the alkoxycarbonyl group represented by R⁷ and R⁸ may be linear, branched, or cyclic, and examples thereof include an alkyl group having 1 to 10 carbon atoms (preferably, 1 to 6 carbon atoms). The alkoxycarbonyl group may further have a substituent.

An alkyl group in the alkylcarbonyl group represented by R⁷ and R⁸ may be linear, branched, or cyclic, and examples thereof include an alkyl group having 1 to 10 carbon atoms (preferably, 1 to 6 carbon atoms). The alkylcarbonyl group may further have a substituent.

The monovalent aliphatic hydrocarbon group having 1 to 10 carbon atoms, represented by R⁷ and R⁸, may be linear, branched, or cyclic.

Examples of the monovalent aliphatic hydrocarbon group having 1 to 10 carbon atoms include an alkyl group having 1 to 10 carbon atoms (preferably having 1 to 6 carbon atoms and more preferably having 1 to 4 carbon atoms), an alkenyl group having 2 to 10 carbon atoms (preferably having 2 to 6 carbon atoms and more preferably having 2 to 4 carbon atoms), and an alkynyl group having 2 to 10 carbon atoms (preferably having 2 to 6 carbon atoms and more preferably having 2 to 4 carbon atoms). In addition, the monovalent aliphatic hydrocarbon group having 1 to 10 carbon atoms may further have a substituent.

Among these, a hydrogen atom is preferable as R⁷ and R⁸.

Rⁱ represents a monovalent substituent.

The monovalent substituent represented by R′ is not particularly limited, and examples thereof include monovalent substituents such as an alkyl group, an alkoxy group, an aryl group, an arylcarbonyloxy group, an aryloxycarbonyl group, an arylamide group, an alkoxycarbonyl group, an alkylcarbonyloxy group, and a cinnamoyloxy group.

The alkyl group, alkoxy group, aryl group, arylcarbonyloxy group, aryloxycarbonyl group, arylamide group, alkoxycarbonyl group, and alkylcarbonyloxy group represented by Rⁱ respectively have the meanings as the alkyl group, alkoxy group, aryl group, arylcarbonyloxy group, aryloxycarbonyl group, arylamide group, alkoxycarbonyl group, and alkylcarbonyloxy group represented by R¹ to R⁶ in General Formula (1) described above, and the suitable aspects are also the same.

As the monovalent substituent represented by Rⁱ, an alkoxy group, an arylcarbonyloxy group, an aryloxycarbonyl group, an arylamide group, or an alkoxycarbonyl group is preferable.

In General Formula (2), in a case where n is 2 or more, a plurality of Rⁱ's may be the same or different from each other.

n represents an integer of 0 to 5.

The number of n is not particularly limited, but is preferably an integer of 0 to 3 and more preferably an integer of 1 to 3.

L¹ represents a single bond or a divalent linking group represented by General Formula (3). In addition, * represents a bonding position to the binaphthyl skeleton in General Formula (1).

In a case where L¹ represents a single bond, the carbon atom represented by C^{A} represents a bonding site to the above-described binaphthyl skeleton in General Formula (1).

Among these, from the viewpoint that a rate of increase in HTP is larger, a single bond is preferable as L¹.

Hereinafter, the divalent linking group represented by General Formula (3) will be described.

*-L²-Ar²—** (3)

In General Formula (3), L² represents a single bond or a divalent linking group.

The divalent linking group represented by L² is not particularly limited, and examples thereof include a divalent aliphatic hydrocarbon group (which may be linear, branched, or cyclic; a divalent aliphatic hydrocarbon group having 1 to 20 carbon atoms is preferable, and examples thereof include an alkylene group, an alkenylene group, and an alkynylene group), —O—, —S—, —SO₂—, —NR^{A}—, —CO— (—C(═O)—), —COO— (—C(═O)O—), —OCO— (—OC(═O)—), —NR^{A}—CO—, —CO—NR^{A}—, —SO₃—, —SO₂NR^{A}—, —NR^{A}SO₂—, —N═N—, —CH═N—, —N═CH—, and a group of a combination of two or more these groups. Here, R^{A} represents a hydrogen atom or an alkyl group (preferably having 1 to 10 carbon atoms).

A hydrogen atom in the above-described divalent linking group may be replaced with another substituent such as a halogen atom.

Among these, as the divalent linking group represented by L², —O—, —CO—, —COO—, or —OCO— is preferable, and —COO— or —OCO— is more preferable.

Ar² represents a divalent aromatic hydrocarbon ring group.

An aromatic hydrocarbon ring constituting the aromatic hydrocarbon ring group is not particularly limited, and examples thereof include an aromatic hydrocarbon ring having 6 to 20 carbon atoms. Among these, an aromatic hydrocarbon ring having 6 to 10 carbon atoms is preferable, and a benzene ring is more preferable.

In General Formula (3), * represents a bonding position to the above-described binaphthyl skeleton in General Formula (1). ** represents a bonding site to the above-described carbon atom represented by C^{A} in General Formula (2).

Next, the monovalent substituent represented by General Formula (4) will be described.

By including a monovalent substituent represented by General Formula (4), the specific compound can have a trans-stilbene derivative structure introduced into the molecule. Specifically, in a case where L³ in the monovalent substituent represented by General Formula (4) represents a single bond, in an olefin double bonding site (C^{B}═C) specified in General Formula (4), Ar³ specified in General Formula (4) and the benzene ring (meaning a benzene ring to which the monovalent substituent represented by General Formula (4) is bonded) included in the binaphthyl skeleton of General Formula (1) have a trans-type arrangement to form a trans-stilbene derivative structure. In addition, in a case where L³ in the monovalent substituent represented by General Formula (4) represents a divalent linking group represented by General Formula (5), in the olefin double bonding site (C^{B}═C) specified in General Formula (4), Ar³ specified in General Formula (4) and Ar⁴ specified in General Formula (5) have a trans-type arrangement to form a trans-stilbene derivative structure.

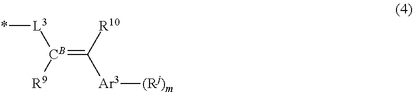

In General Formula (4), Ar³ represents an (m+1)-valent aromatic hydrocarbon ring group.

An aromatic hydrocarbon ring constituting the aromatic hydrocarbon ring group is not particularly limited, and examples thereof include an aromatic hydrocarbon ring having 6 to 20 carbon atoms. Among these, an aromatic hydrocarbon ring having 6 to 10 carbon atoms is preferable, and a benzene ring is more preferable.

C^{B} represents a carbon atom.

R⁹ and R¹⁰ each independently represent a hydrogen atom, a cyano group, a substituted boryl group, a substituted silyl group, a substituted aluminum group, a halogen atom, an alkoxycarbonyl group, an alkylcarbonyl group, or a monovalent aliphatic hydrocarbon group having 1 to 10 carbon atoms.

Examples of the substituted boryl group, the substituted silyl group, the substituted aluminum group, the halogen atom, the alkoxycarbonyl group, the alkylcarbonyl group, and the monovalent aliphatic hydrocarbon group having 1 to 10 carbon atoms, represented by R⁹ and R¹⁰, include the same groups as the substituted boryl group, the substituted silyl group, the substituted aluminum group, the halogen atom, the alkoxycarbonyl group, the alkylcarbonyl group, and the monovalent aliphatic hydrocarbon group having 1 to 10 carbon atoms, represented by R⁷ and R⁸ in General Formula (2), and the suitable aspects thereof are also the same.

Among these, a hydrogen atom is preferable as R⁹ and R¹⁰.

R^{j} represents a monovalent substituent.

Examples of the monovalent substituent represented by R^{j} include the same group as the monovalent substituent represented by R′ in General Formula (2), and the suitable aspect thereof is also the same.

In General Formula (4), in a case where m is 2 or more, a plurality of R^{j}'s may be the same or different from each other.

m represents an integer of 0 to 5.

The number of m is not particularly limited, but is preferably an integer of 0 to 3 and more preferably an integer of 1 to 3.

L³ represents a single bond or a divalent linking group represented by General Formula (5). In addition, * represents a bonding position to the binaphthyl skeleton in General Formula (1).

In a case where L³ represents a single bond, the carbon atom represented by C^{B} represents a bonding site to the above-described binaphthyl skeleton in General Formula (1).

Among these, a single bond is preferable as L³.

Hereinafter, a divalent linking group represented by General Formula (5) will be described.

*-L⁴-Ar⁴—** (5)

In General Formula (5), L⁴ represents a single bond or a divalent linking group.

Examples of the divalent linking group represented by L⁴ include the same group as the divalent linking group represented by L² in General Formula (3), and the suitable aspect thereof is also the same.

Ar⁴ represents a divalent aromatic hydrocarbon ring group.

Examples of the divalent aromatic hydrocarbon ring group represented by Ar⁴ include the same group as the divalent aromatic hydrocarbon ring group represented by Ar² in General Formula (3), and the suitable aspect thereof is also the same.

In General Formula (5), * represents a bonding position to the above-described binaphthyl skeleton in General Formula (1). ** represents a bonding site to the above-described carbon atom represented by C^{B} in General Formula (4).

In General Formula (1), from the viewpoint that the rate of increase in HTP is larger, as the monovalent substituent represented by R¹ to R⁶, a group other than the monovalent substituent represented by General Formula (4) is preferable.

In addition, from the viewpoint that HTP after exposure is large and the rate of increase in HTP is larger, it is preferable that two or more of R¹ to R⁶ in General Formula (1) are the above-described monovalent substituent represented by General Formula (2). Among these, it is preferable that one or more groups selected from the group consisting of R′, R³, and R⁵ represent the monovalent substituent represented by General Formula (2) and one or more groups selected from the group consisting of R², R⁴, and R⁶ represent the monovalent substituent represented by General Formula (2).

In addition, from the viewpoint that HTP after exposure is larger, it is preferable that the specific compound satisfies one or more aspects selected from the following aspect (A), the following aspect (B), and the following aspect (C).

Aspect (A): in the monovalent substituent represented by General Formula (2), L′ represents the divalent linking group represented by General Formula (3).

Aspect (B): in the monovalent substituent represented by General Formula (2), R′ represents *-L^{S1}-monovalent aromatic hydrocarbon ring group.

Aspect (C): R¹ and R² in General Formula (1) are bonded to each other to represent *-L^{S2}-divalent aromatic hydrocarbon ring group-L^{S2}-*.

L^{S1} and L^{S2} each independently represent a single bond or a divalent linking group. The divalent linking group represented by L^{S1} and L^{S2} has the same meaning as the divalent linking group represented by L² in General Formula (3). As L^{S1} and L^{S2}, a single bond, a divalent aliphatic hydrocarbon group (which may be linear, branched, or cyclic; a divalent aliphatic hydrocarbon group having 1 to 20 carbon atoms is preferable, and examples thereof include an alkylene group, an alkenylene group, and an alkynylene group), —O—, —CO—, —NH—CO—, —CO—NH—, —COO—, or —OCO— is preferable.

An aromatic hydrocarbon ring constituting the monovalent aromatic hydrocarbon ring group shown in the aspect (B) is not particularly limited, and examples thereof include an aromatic hydrocarbon ring having 6 to 20 carbon atoms. Among these, an aromatic hydrocarbon ring having 6 to 10 carbon atoms is preferable, and a benzene ring is more preferable.

An aromatic hydrocarbon ring constituting the divalent aromatic hydrocarbon ring group shown in the aspect (C) is not particularly limited, and examples thereof include an aromatic hydrocarbon ring having 6 to 20 carbon atoms. Among these, an aromatic hydrocarbon ring having 6 to 10 carbon atoms is preferable, and a benzene ring is more preferable.

In particular, from the viewpoint that HTP after exposure is larger, it is preferable that the specific compound is represented by General Formula (1-2) described later, and satisfies one or more aspects selected from the above-described aspect (A), the above-described aspect (B), and the above-described aspect (C).

In a case of the above-described configuration, the specific compound includes a structure in which three or more aromatic hydrocarbon ring groups are linked to each other through a single bond or a divalent linking group (however, the linking of naphthalene rings in the binaphthyl skeleton is not included), and it is assumed that this structure is one of the reasons that HTP after exposure is larger.

In detail, the specific compound of the aspect (A) includes a structure in which the naphthalene ring included in the binaphthyl skeleton, Ar² specified in General Formula (3), and Ar′ specified in General Formula (2) are linked to each other through a single bond or a divalent linking group. In addition, the specific compound of the aspect (B) includes a structure in which the naphthalene ring included in the binaphthyl skeleton, Ar′ specified in General Formula (2), and the aromatic hydrocarbon ring in R′ are linked to each other through a single bond or a divalent linking group. In addition, the specific compound of the aspect (C) includes a structure in which two naphthalene rings included in the binaphthyl skeleton, and the aromatic hydrocarbon ring in *-L^{S2}-divalent aromatic hydrocarbon ring group -L^{S2}-* formed by bonding R¹ and R² to each other are linked to each other through a single bond or a divalent linking group.

In addition, from the viewpoint that HTP after exposure is large and the temperature dependence of HTP is lower, it is preferable that R¹ and R² in General Formula (1) are bonded to each other to form a ring structure.

The ring structure is not particularly limited and may be either an aromatic ring or a non-aromatic ring, but is preferably a non-aromatic ring and may include a hetero atom such as a nitrogen atom, an oxygen atom, and a sulfur atom.

The number of ring members of the ring structure is not particularly limited, and for example, is 5 to 12. The number of ring members is a number including four carbon atoms specified in General Formula (1).

In General Formula (1), a portion where the solid line and the broken line are parallel to each other represents a single bond or a double bond. In a case where the portion where the solid line and the broken line are parallel to each other is a single bond, the specific compound corresponds to a compound represented by General Formula (1-1), and in a case where the portion where the solid line and the broken line are parallel to each other is a double bond, the specific compound corresponds to a compound represented by General Formula (1-2). Among these, from the viewpoint that the effects of the present invention are more excellent, the specific compound is preferably the compound represented by General Formula (1-2).

R¹ to R⁶ in General Formula (1-1) and General Formula (1-2) respectively have the same meanings as R¹ to R⁶ in General Formula (1), and the suitable aspects are also the same.

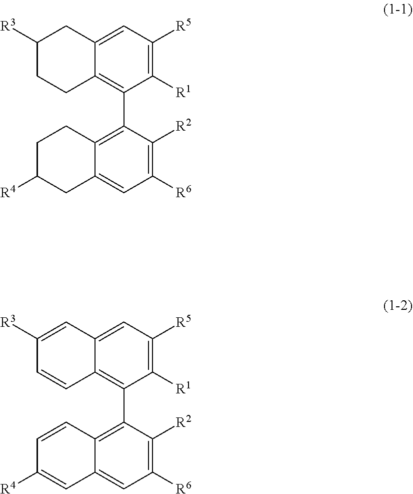

Among these, the specific compound is preferably a compound represented by General Formula (X1).

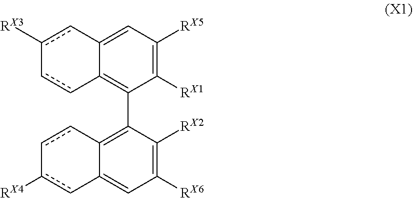

In General Formula (X1), R^{X1} to R^{X6} each independently represent a hydrogen atom or a monovalent substituent. However, at least one of R^{X1}, R^{X2}, R^{X3}, R^{X4}, R^{X5}, or R^{X6} represents the above-described monovalent substituent represented by General Formula (2). In addition, R^{X1} to R^{X6} do not include the above-described monovalent substituent represented by General Formula (4).

Examples of the monovalent substituent represented by R^{X1} to R^{X6} include monovalent substituents such as an alkyl group, an alkoxy group, an aryl group, an arylcarbonyloxy group, an aryloxycarbonyl group, an arylamide group, an alkoxycarbonyl group, an alkylcarbonyloxy group, and a cinnamoyloxy group; and the above-described monovalent substituent represented by General Formula (2).

The alkyl group, alkoxy group, aryl group, arylcarbonyloxy group, aryloxycarbonyl group, arylamide group, alkoxycarbonyl group, and alkylcarbonyloxy group represented by R^{X1} to R^{X6} respectively have the meanings as the alkyl group, alkoxy group, aryl group, arylcarbonyloxy group, aryloxycarbonyl group, arylamide group, alkoxycarbonyl group, and alkylcarbonyloxy group represented by R¹ to R⁶ in General Formula (1) described above, and the suitable aspects are also the same.

Among these, as the monovalent substituent represented by R^{X1} to R^{X6}, an alkoxy group, an arylcarbonyloxy group, an aryloxycarbonyl group, an arylamide group, or the above-described monovalent substituent represented by General Formula (2) is preferable. In addition, as described later, R^{X1} and R^{X2} may be bonded to each other to form a ring structure.

From the viewpoint that HTP after exposure is large and the rate of increase in HTP is larger, it is preferable that two or more of R^{X1} to R^{X6} in General Formula (X1) are the above-described monovalent substituent represented by General Formula (2). Among these, it is preferable that one or more groups selected from the group consisting of R^{X1}, R^{X3}, and R^{X5} represent the monovalent substituent represented by General Formula (2) and one or more groups selected from the group consisting of R^{X2}, R^{X4}, and R^{X6} represent the monovalent substituent represented by General Formula (2).

In addition, from the viewpoint that HTP after exposure is larger, it is preferable that the compound represented by General Formula (X1) satisfies one or more aspects selected from the following aspect (A), the following aspect (B), and the following aspect (C).

Aspect (A): in the monovalent substituent represented by General Formula (2), L′ represents the above-described divalent linking group represented by General Formula (3).

Aspect (B): in the monovalent substituent represented by General Formula (2), R′ represents *-L^{S1}-monovalent aromatic hydrocarbon ring group.

Aspect (C): R¹ and R² in General Formula (1) are bonded to each other to represent *-L^{S2}-divalent aromatic hydrocarbon ring group-L^{S2}-*.

L^{S1} and L^{S2} each independently represent a single bond or a divalent linking group. The divalent linking group represented by L^{S1} and L^{S2} has the same meaning as the divalent linking group represented by L² in General Formula (3). As L^{S1} and L^{S2}, a single bond, a divalent aliphatic hydrocarbon group (which may be linear, branched, or cyclic; a divalent aliphatic hydrocarbon group having 1 to 20 carbon atoms is preferable, and examples thereof include an alkylene group, an alkenylene group, and an alkynylene group), —O—, —CO—, —NH—CO—, —CO—NH—, —COO—, or —OCO— is preferable.

An aromatic hydrocarbon ring constituting the monovalent aromatic hydrocarbon ring group shown in the aspect (B) is not particularly limited, and examples thereof include an aromatic hydrocarbon ring having 6 to 20 carbon atoms. Among these, an aromatic hydrocarbon ring having 6 to 10 carbon atoms is preferable, and a benzene ring is more preferable.

An aromatic hydrocarbon ring constituting the divalent aromatic hydrocarbon ring group shown in the aspect (C) is not particularly limited, and examples thereof include an aromatic hydrocarbon ring having 6 to 20 carbon atoms. Among these, an aromatic hydrocarbon ring having 6 to 10 carbon atoms is preferable, and a benzene ring is more preferable.

In particular, from the viewpoint that HTP after exposure is larger, it is preferable that the compound represented by General Formula (X1) has the same structure as the structure represented by General Formula (1-2) described above (in other words, all of the portions in General Formula (X1), where the solid line and the broken line are parallel to each other, represent a double bond), and satisfies one or more aspects selected from the above-described aspect (A), the above-described aspect (B), and the above-described aspect (C).

In a case of the above-described configuration, the compound represented by General Formula (X1) includes a structure in which three or more aromatic hydrocarbon ring groups are linked to each other through a single bond or a divalent linking group (however, the linking of naphthalene rings in the binaphthyl skeleton is not included), and it is assumed that this structure is one of the reasons that HTP after exposure is larger. In detail, the compound represented by General Formula (X1) of the aspect (A) includes a structure in which the naphthalene ring included in the binaphthyl skeleton, Ar² specified in General Formula (3), and Ar¹ specified in General Formula (2) are linked to each other through a single bond or a divalent linking group. In addition, the compound represented by General Formula (X1) of the aspect (B) includes a structure in which the naphthalene ring included in the binaphthyl skeleton, Ar′ specified in General Formula (2), and the aromatic hydrocarbon ring in R′ are linked to each other through a single bond or a divalent linking group. In addition, the compound represented by General Formula (X1) of the aspect (C) includes a structure in which two naphthalene rings included in the binaphthyl skeleton, and the aromatic hydrocarbon ring in *-L^{S2}-divalent aromatic hydrocarbon ring group -L^{S2}-* formed by bonding R¹ and R² to each other are linked to each other through a single bond or a divalent linking group.

In addition, from the viewpoint that HTP after exposure is large and the temperature dependence of HTP is lower, it is preferable that R^{X1} and R^{X2} in General Formula (X1) are bonded to each other to form a ring structure.

The ring structure is not particularly limited and may be either an aromatic ring or a non-aromatic ring, but is preferably a non-aromatic ring and may include a hetero atom such as a nitrogen atom, an oxygen atom, and a sulfur atom.

The number of ring members of the ring structure is not particularly limited, and for example, is 5 to 12. The number of ring members is a number including four carbon atoms specified in General Formula (X1).

In the specific compound, the content (hereinafter, also referred to as “X1 (mole)”) of the above-described monovalent substituent represented by General Formula (2) and the content (hereinafter, also referred to as “Y1 (mole)”) of the above-described monovalent substituent represented by General Formula (4) can be obtained by ¹H nuclear magnetic resonance (NMR). In a case where the specific compound includes the above-described monovalent substituent represented by General Formula (2) and the above-described monovalent substituent represented by General Formula (4), X1/Y1 is preferably 5 or more and more preferably 10 or more.

The specific compound can be synthesized according to a known method. The specific compound can be synthesized by, for example, a producing method including a step of reducing a binaphthol derivative having a tolan structure by catalytic reduction, and a producing method including a step of converting a binaphthol derivative having a tolan structure into a substituted alkene by hydrometalization.

In addition, the specific compound may be an R-form or an S-form, or may be a mixture of R-form and S-form.

Specific examples of the above-described specific compound are described below, but the specific compound is not limited thereto. In the following compounds, only R-form or only S-form may be exemplified, but the corresponding S-form and R-form can also be used.

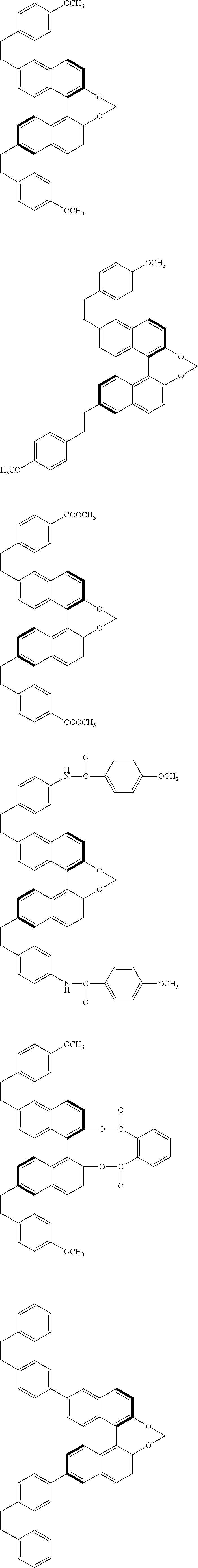

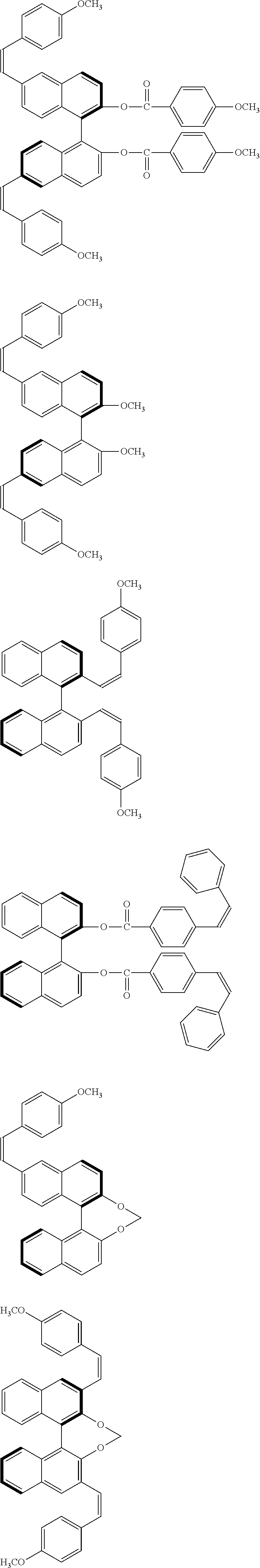

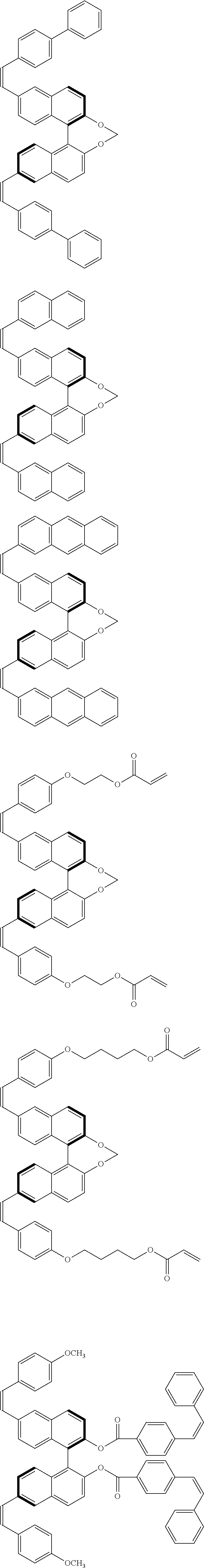

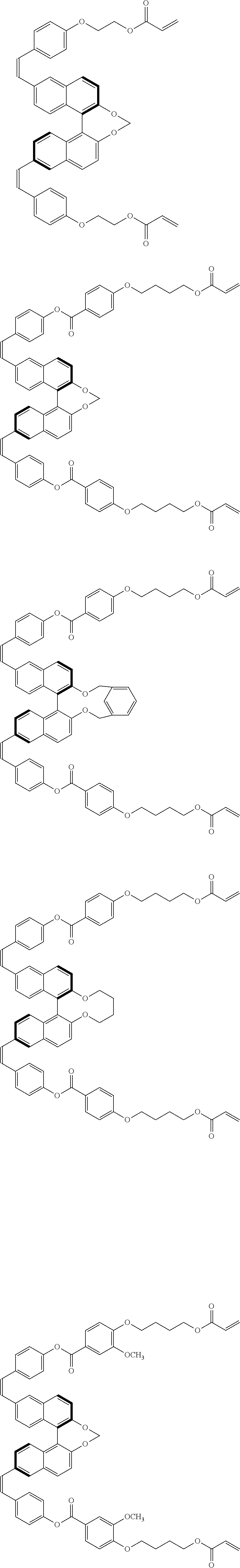

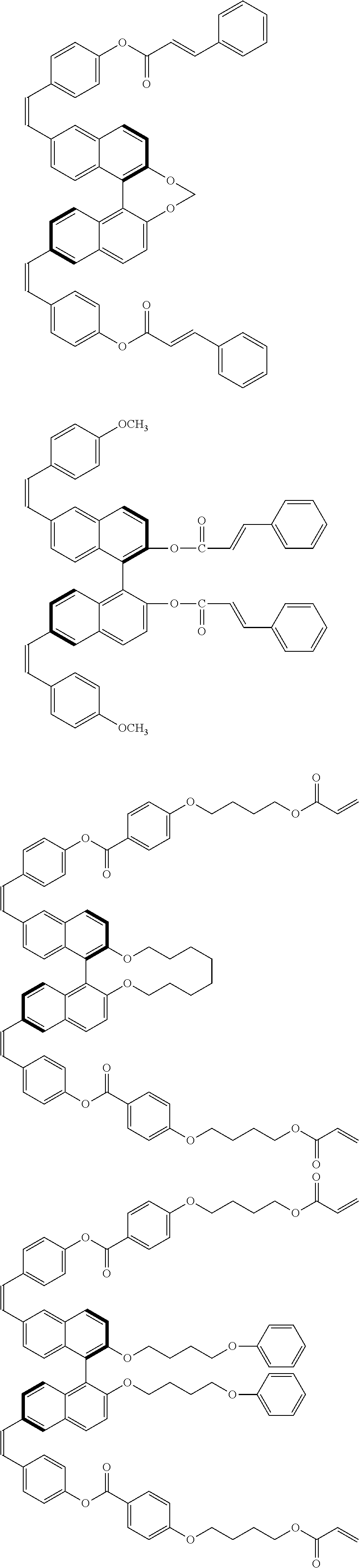

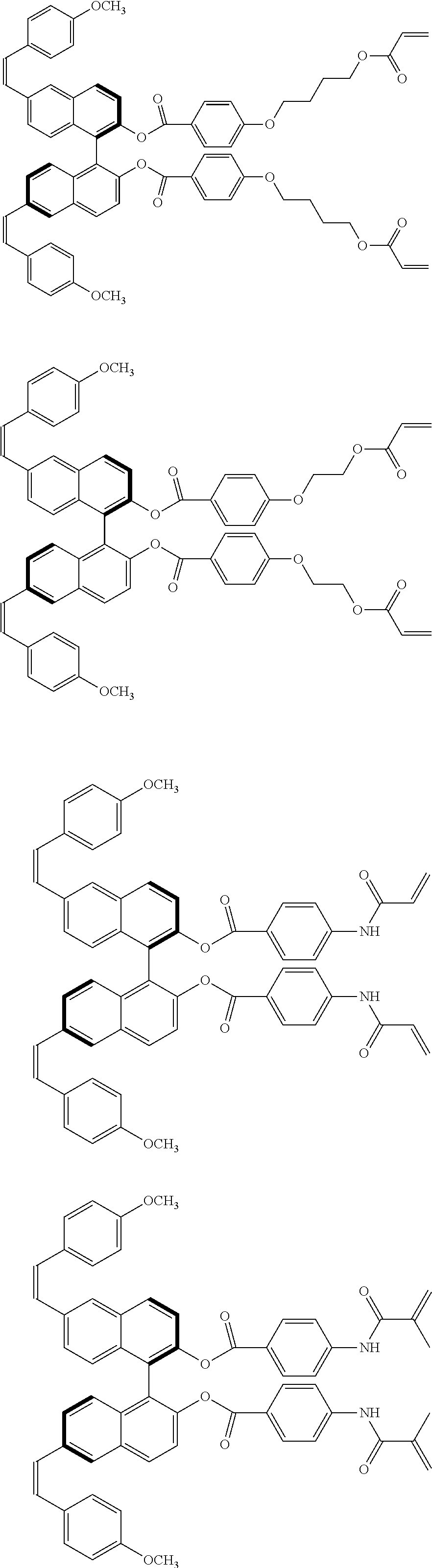

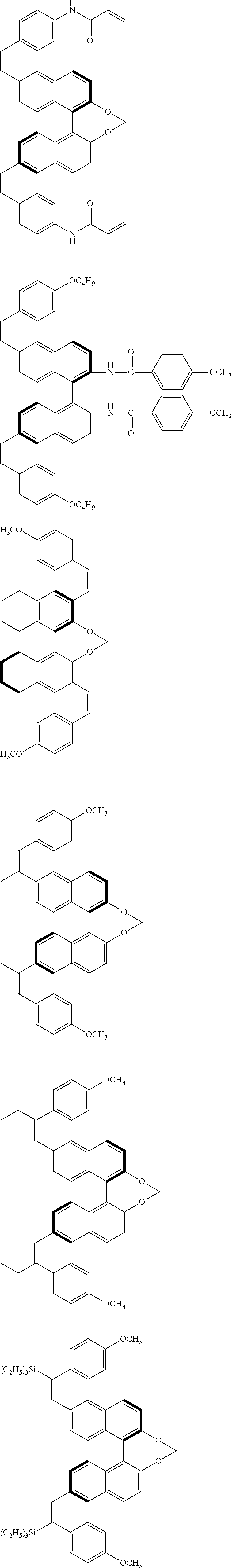

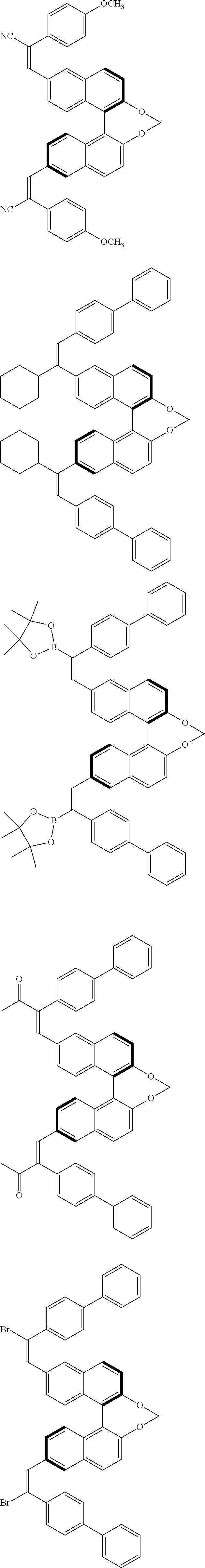

[Uses]

The above-described specific compound can be applied to various uses as a so-called chiral agent. For example, by using a liquid crystal composition obtained by mixing the specific compound and a liquid crystalline compound, a cholesteric liquid crystalline phase can be formed.

[Mixture]

A mixture according to an embodiment of the present invention consists of the above-described compound represented by General Formula (X1) and a compound represented by General Formula (Y1).

Hereinafter, the compound represented by General Formula (Y1) will be described in detail.

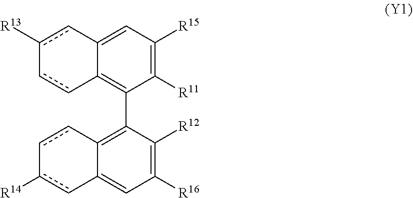

In General Formula (Y1), R¹¹ to R¹⁶ each independently represent a hydrogen atom or a monovalent substituent. However, at least one of R¹¹, R¹², R¹³, R¹⁴, R¹⁵, or R¹⁶ represents a monovalent substituent represented by General Formula (6) described later.

Examples of the monovalent substituent represented by R¹¹ to R¹⁶ include monovalent substituents such as an alkyl group, an alkoxy group, an aryl group, an arylcarbonyloxy group, an aryloxycarbonyl group, an arylamide group, an alkoxycarbonyl group, an alkylcarbonyloxy group, and a cinnamoyloxy group; the above-described monovalent substituent represented by General Formula (2); and a monovalent substituent represented by General Formula (6) described later.

The alkyl group, alkoxy group, aryl group, arylcarbonyloxy group, aryloxycarbonyl group, arylamide group, alkoxycarbonyl group, and alkylcarbonyloxy group represented by R¹¹ to R¹⁶ respectively have the meanings as the alkyl group, alkoxy group, aryl group, arylcarbonyloxy group, aryloxycarbonyl group, arylamide group, alkoxycarbonyl group, and alkylcarbonyloxy group represented by R¹ to R⁶ in General Formula (1) described above, and the suitable aspects are also the same.

Among these, as the monovalent substituent represented by R¹¹ to R¹⁶, an alkoxy group, an arylcarbonyloxy group, an aryloxycarbonyl group, an arylamide group, or a monovalent substituent represented by General Formula (6) described above is preferable. In addition, as described later, R¹¹ and R¹² may be bonded to each other to form a ring structure.

Hereinafter, the monovalent substituent represented by General Formula (6) will be described.

By including a monovalent substituent represented by General Formula (6), the compound represented by General Formula (Y1) has a trans-stilbene derivative structure introduced into the molecule. Specifically, in a case where L⁵ in the monovalent substituent represented by General Formula (6) represents a single bond, in an olefin double bonding site (C^{C}═C) specified in General Formula (6), Ar⁵ specified in General Formula (6) and the benzene ring (meaning a benzene ring to which the monovalent substituent represented by General Formula (6) is bonded) included in the binaphthyl skeleton of General Formula (Y1) have a trans-type arrangement to form a trans-stilbene derivative structure. In addition, in a case where L⁵ in the monovalent substituent represented by General Formula (6) represents a divalent linking group represented by General Formula (7), in the olefin double bonding site (C^{C}═C) specified in General Formula (6), Ar⁵ specified in General Formula (6) and Ar⁶ specified in General Formula (7) have a trans-type arrangement to form a trans-stilbene derivative structure.

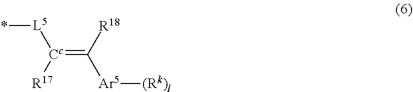

In General Formula (6), Ar⁵ represents an (1+1)-valent aromatic hydrocarbon ring group.

An aromatic hydrocarbon ring constituting the aromatic hydrocarbon ring group is not particularly limited, and examples thereof include an aromatic hydrocarbon ring having 6 to 20 carbon atoms. Among these, an aromatic hydrocarbon ring having 6 to 10 carbon atoms is preferable, and a benzene ring is more preferable.

C^{C} represents a carbon atom.

R¹⁷ and R¹⁸ each independently represent a hydrogen atom, a cyano group, a substituted boryl group, a substituted silyl group, a substituted aluminum group, a halogen atom, an alkoxycarbonyl group, an alkylcarbonyl group, or a monovalent aliphatic hydrocarbon group having 1 to 10 carbon atoms.

Examples of the substituted boryl group, the substituted silyl group, the substituted aluminum group, the halogen atom, the alkoxycarbonyl group, the alkylcarbonyl group, and the monovalent aliphatic hydrocarbon group having 1 to 10 carbon atoms, represented by R¹⁷ and R¹⁸, include the same groups as the substituted boryl group, the substituted silyl group, the substituted aluminum group, the halogen atom, the alkoxycarbonyl group, the alkylcarbonyl group, and the monovalent aliphatic hydrocarbon group having 1 to 10 carbon atoms, represented by R⁷ and R⁸ in General Formula (2), and the suitable aspects thereof are also the same.

Among these, a hydrogen atom is preferable as R¹⁷ and R¹⁸.

R^{k} represents a monovalent substituent.

Examples of the monovalent substituent represented by R^{k} include the same group as the monovalent substituent represented by Rⁱ in General Formula (2), and the suitable aspect thereof is also the same.

In General Formula (6), in a case where 1 is 2 or more, a plurality of R^{k}'s may be the same or different from each other.

1 represents an integer of 0 to 5.

The number of 1 is not particularly limited, but is preferably an integer of 0 to 3 and more preferably an integer of 1 to 3.

L⁵ represents a single bond or a divalent linking group represented by General Formula (7). In addition, * represents a bonding position to the above-described binaphthyl skeleton in General Formula (Y1).

In a case where L⁵ represents a single bond, the carbon atom represented by C^{C} represents a bonding site to the above-described binaphthyl skeleton in General Formula (Y1).

Among these, a single bond is preferable as L⁵.

Hereinafter, the divalent linking group represented by General Formula (7) will be described.

*-L⁶-Ar⁶—** (7)

In General Formula (7), L⁶ represents a single bond or a divalent linking group.

Examples of the divalent linking group represented by L⁶ include the same group as the divalent linking group represented by L² in General Formula (3), and the suitable aspect thereof is also the same.

Ar⁶ represents a divalent aromatic hydrocarbon ring group.

Examples of the divalent aromatic hydrocarbon ring group represented by Ar⁶ include the same group as the divalent aromatic hydrocarbon ring group represented by Ar² in General Formula (3), and the suitable aspect thereof is also the same.

In General Formula (7), * represents a bonding position to the above-described binaphthyl skeleton in General Formula (Y1). ** represents a bonding site to C^{C} in General Formula (6) described above.

In addition, it is preferable that two or more of R¹¹ to R¹⁶ in General Formula (Y1) are the above-described monovalent substituent represented by General Formula (6). Among these, it is preferable that one or more groups selected from the group consisting of R¹¹, R¹³, and R¹⁵ represent the monovalent substituent represented by General Formula (6) and one or more groups selected from the group consisting of R¹², R¹⁴, and R¹⁶ represent the monovalent substituent represented by General Formula (6).

In addition, from the viewpoint that the temperature dependence of HTP is lower, it is preferable that R¹¹ and R¹² in General Formula (Y1) are bonded to each other to form a ring structure.

The ring structure is not particularly limited and may be either an aromatic ring or a non-aromatic ring, but is preferably a non-aromatic ring and may include a hetero atom such as a nitrogen atom, an oxygen atom, and a sulfur atom.

The number of ring members of the ring structure is not particularly limited, and for example, is 5 to 12. The number of ring members is a number including four carbon atoms specified in General Formula (Y1).

In General Formula (Y1), a portion where the solid line and the broken line are parallel to each other represents a single bond or a double bond. In a case where the portion where the solid line and the broken line are parallel to each other is a single bond, the compound represented by General Formula (Y1) corresponds to a compound represented by General Formula (Y1-1), and in a case where the portion where the solid line and the broken line are parallel to each other is a double bond, the compound represented by General Formula (Y1) corresponds to a compound represented by General Formula (Y1-2).

R¹¹ to R¹⁶ in General Formula (Y1-1) and General Formula (Y1-2) respectively have the same meanings as R¹¹ to R¹⁶ in General Formula (Y1), and the suitable aspects are also the same.

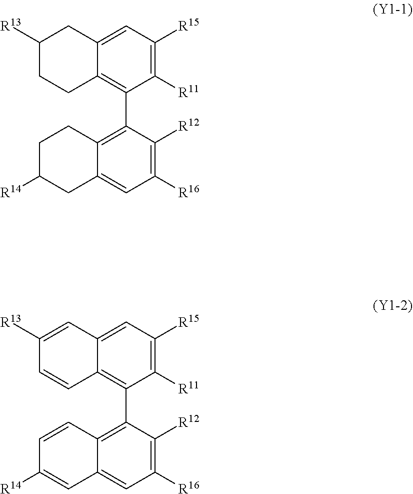

In the mixture, the mixing ratio of the compound represented by General Formula (X1) and the compound represented by General Formula (Y1) is not particularly limited, and it is sufficient that the compounds are mixed with each other at an arbitrary proportion so as to obtain a desired initial HTP and rate of increase in HTP. In other words, in the above-described mixture, by adjusting the mixing ratio of the compound represented by General Formula (X1) and the compound represented by General Formula (Y1), a desired initial HTP and rate of increase in HTP can be set.

Among these, from the viewpoint that the rate of increase in HTP is larger, the content ratio of the content (hereinafter, also referred to as “X2 (mole)”) of the monovalent substituent represented by General Formula (2) to the content (hereinafter, also referred to as “Y2 (mole)”) of the monovalent substituent represented by General Formula (6) in the mixture (content (X2/Y2) of the monovalent substituent represented by General Formula (2) with respect to the content of the monovalent substituent represented by General Formula (6)) is preferably 5 or more and more preferably 10 or more.

In the mixture, the content of the above-described monovalent substituent represented by General Formula (2) and the content of the monovalent substituent represented by General Formula (6) can be obtained by ¹H nuclear magnetic resonance (NMR).

In addition, in the mixture, as a combination of the compound represented by General Formula (X1) and the compound represented by General Formula (Y1), it is preferable that the combination satisfies both the following requirement (A) and the following requirement (B).

Requirement (A):

in the mixture, all of the portions in General Formula (X1) and General Formula (Y1), where the solid line and the broken line are parallel to each other, represent a double bond, or all of the portions in General Formula (X1) and General Formula (Y1), where the solid line and the broken line are parallel to each other, represent a single bond.

Requirement (B):

In the compound represented by General Formula (X1), in a case where R^{X1} and R^{X2} are not bonded to each other to form a ring structure:

in the mixture, in a case where R^{X1} in the compound represented by General Formula (X1) represents the monovalent substituent represented by General Formula (2), R¹¹ in the compound represented by General Formula (Y1) represents the monovalent substituent represented by General Formula (6); in a case where R^{X2} in the compound represented by General Formula (X1) represents the monovalent substituent represented by General Formula (2), R¹² in the compound represented by General Formula (Y1) represents the monovalent substituent represented by General Formula (6); in a case where R^{X3} in the compound represented by General Formula (X1) represents the monovalent substituent represented by General Formula (2), R¹³ in the compound represented by General Formula (Y1) represents the monovalent substituent represented by General Formula (6); in a case where R^{X4} in the compound represented by General Formula (X1) represents the monovalent substituent represented by General Formula (2), R¹⁴ in the compound represented by General Formula (Y1) represents the monovalent substituent represented by General Formula (6); in a case where R^{X5} in the compound represented by General Formula (X1) represents the monovalent substituent represented by General Formula (2), R¹⁵ in the compound represented by General Formula (Y1) represents the monovalent substituent represented by General Formula (6); and in a case where R^{X6} in the compound represented by General Formula (X1) represents the monovalent substituent represented by General Formula (2), R¹⁶ in the compound represented by General Formula (Y1) represents the monovalent substituent represented by General Formula (6).

furthermore, in the above-described aspects, it is preferable that L¹, R⁷, R⁸, Ar¹, Rⁱ, and n in General Formula (2) are the same as L⁵, R¹⁷, R¹⁸, Ar⁵, R^{k}, and 1 in General Formula (6) respectively, and R^{X1} to R^{X6} in the compound represented by General Formula (X1), representing other than the monovalent substituent represented by General Formula (2), are the same as R¹¹ to R¹⁶ in the compound represented by General Formula (Y1), representing other than the monovalent substituent represented by General Formula (6).

In the compound represented by General Formula (X1), in a case where R^{X1} and R^{X2} are bonded to each other to form a ring structure:

in the mixture, in a case where R^{X3} in the compound represented by General Formula (X1) represents the monovalent substituent represented by General Formula (2), R¹³ in the compound represented by General Formula (Y1) represents the monovalent substituent represented by General Formula (6); in a case where R^{X4} in the compound represented by General Formula (X1) represents the monovalent substituent represented by General Formula (2), R¹⁴ in the compound represented by General Formula (Y1) represents the monovalent substituent represented by General Formula (6); in a case where R^{X5} in the compound represented by General Formula (X1) represents the monovalent substituent represented by General Formula (2), R¹⁵ in the compound represented by General Formula (Y1) represents the monovalent substituent represented by General Formula (6); in a case where R^{X6} in the compound represented by General Formula (X1) represents the monovalent substituent represented by General Formula (2), R¹⁶ in the compound represented by General Formula (Y1) represents the monovalent substituent represented by General Formula (6); and R¹¹ and R¹² in the compound represented by General Formula (Y1) are bonded to each other to represent a ring structure.

furthermore, in the above-described aspects, it is preferable that L¹, R⁷, R⁸, Ar¹, Rⁱ, and n in General Formula (2) are the same as L⁵, R¹⁷, R¹⁸, Ar⁵, R^{k}, and 1 in General Formula (6) respectively; R^{X3} to R^{X6} in the compound represented by General Formula (X1), representing other than the monovalent substituent represented by General Formula (2), are the same as R¹³ to R¹⁶ in the compound represented by General Formula (Y1), representing other than the monovalent substituent represented by General Formula (6); and the ring structure formed by bonding R^{X1} and R^{X2} in the compound represented by General Formula (X1) to each other is the same as the ring structure formed by bonding R¹¹ and R¹² in the compound represented by General Formula (Y1) to each other.

[Uses]

The above-described mixture can be applied to various uses as a so-called chiral agent. For example, by using a liquid crystal composition obtained by mixing the mixture and a liquid crystalline compound, a cholesteric liquid crystalline phase can be formed.

[Liquid Crystal Composition]

Next, a liquid crystal composition according to an embodiment of the present invention will be described.

The liquid crystal composition according to an embodiment of the present invention includes a liquid crystalline compound, and the above-described specific compound or the above-described mixture.

Hereinafter, respective components included in the liquid crystal composition according to the embodiment of the present invention will be described.

[Liquid Crystalline Compound]

The liquid crystalline compound means a compound exhibiting liquid crystallinity. For the compound exhibiting liquid crystallinity, it is intended that the compound has properties of expressing a mesophase between a crystalline phase (low temperature side) and an isotropic phase (high temperature side) in a case of changing a temperature. As a specific observation method, optical anisotropy and fluidity derived from a liquid crystalline phase can be confirmed by performing an observation using a polarizing microscope while heating the compound or lowering a temperature of the compound with a hot stage system FP90, manufactured by METTLER TOLEDO, or the like.

The liquid crystalline compound is not particularly limited as long as it has liquid crystallinity, and examples thereof include a rod-like nematic liquid crystalline compound.

Examples of the rod-like nematic liquid crystalline compound include azomethines, azoxys, cyanobiphenyls, cyanophenyl esters, benzoic acid esters, cyclohexanecarboxylic acid phenyl esters, cyanophenylcyclohexanes, cyano-substituted phenylpyrimidines, alkoxy-substituted phenylpyrimidines, phenyldioxanes, tolans, and alkenylcyclohexylbenzonitriles. High-molecular-weight liquid crystalline compounds can also be used as well as low-molecular-weight liquid crystalline compounds.

The liquid crystalline compound may be polymerizable or non-polymerizable.

Rod-like liquid crystalline compounds having no polymerizable group are described in various documents (for example, Y. Goto et al., Mol. Cryst. Liq. Cryst. 1995, Vol. 260, pp. 23 to 28).

Meanwhile, a polymerizable rod-like liquid crystalline compound is obtained by introducing a polymerizable group into the rod-like liquid crystalline compound. Examples of the polymerizable group include an unsaturated polymerizable group, an epoxy group, and an aziridinyl group. Among these, an unsaturated polymerizable group is preferable and an ethylenically unsaturated polymerizable group is more preferable. The polymerizable group can be introduced into the molecule of the rod-like liquid crystalline compound by various methods. The number of polymerizable groups included in the polymerizable rod-like liquid crystalline compound is preferably 1 to 6 and more preferably 1 to 3. Two or more kinds of polymerizable rod-like liquid crystalline compounds may be used in combination. In a case of using two or more kinds of polymerizable rod-like liquid crystalline compounds in combination, the alignment temperature can be lowered.

From the viewpoint that the cholesteric liquid crystalline phase can be immobilized, as the liquid crystalline compound, a liquid crystalline compound having at least one or more polymerizable groups is preferable, and a liquid crystalline compound having at least two or more polymerizable groups is more preferable.

As the liquid crystalline compound, a compound represented by General Formula (LC) is preferable.

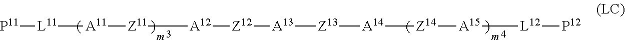

In General Formula (LC), P¹¹ and P¹² each independently represent a hydrogen atom or a polymerizable group. However, at least one of P¹¹ or P¹² represents a polymerizable group. L¹¹ and L¹² each independently represent a single bond or a divalent linking group. A¹¹ to A¹⁵ each independently represent an aromatic hydrocarbon ring group or aromatic heterocyclic group which may have a substituent. Z¹¹ to Z¹⁴ each independently represent a single bond or a divalent linking group. m³ and m⁴ each independently represent an integer of 0 or 1.

In General Formula (LC), the polymerizable group represented by P¹¹ and 1³¹² is not particularly limited, and examples thereof include the polymerizable group represented by General Formulae (P-1) to (P-21) described above.

At least one or more of P¹¹ or P¹² represent a polymerizable group, and it is preferable that both of P¹¹ and P¹² represent a polymerizable group.

In General Formula (LC), the divalent linking group represented by L¹¹ and L¹² is not particularly limited, and examples thereof include a linear or branched alkylene group having 1 to 20 carbon atoms, and a linking group selected from the group consisting of a linear or branched alkylene group having 1 to 20 carbon atoms, in which one or two or more —CH₂— is replaced with —O—, —S—, —NH—, —N(CH₃)—, —CO—, —OCO—, or —COO—. As the divalent linking group represented by L¹¹ and L¹², a group of a linear or branched alkylene group having 1 to 20 carbon atoms, in which one or two or more —CH₂— is replaced with —O— is preferable.

In General Formula (LC), A¹¹ to A¹⁵ each independently represent an aromatic hydrocarbon ring group or aromatic heterocyclic group which may have a substituent.

The number of ring members in the above-described aromatic hydrocarbon ring group is not particularly limited, but is, for example, 5 to 10.

The aromatic hydrocarbon ring constituting the aromatic hydrocarbon ring group may have a monocyclic structure or a polycyclic structure.

The number of carbon atoms in the above-described aromatic hydrocarbon ring is not particularly limited, but is preferably 6 to 18 and more preferably 6 to 10. Specific examples of the aromatic hydrocarbon ring include a benzene ring, a biphenyl ring, a naphthalene ring, an anthracene ring, a phenanthrene ring, and a fluorene ring. Among these, a benzene ring is preferable. The above-described aromatic hydrocarbon ring constitutes an aromatic hydrocarbon ring group by removing two hydrogen atoms on the ring.

The number of ring members in the above-described aromatic heterocyclic group is, for example, 5 to 10.

The aromatic hetero ring constituting the aromatic heterocyclic group may have a monocyclic structure or a polycyclic structure.

Examples of a hetero atom included in the above-described aromatic heterocyclic group include a nitrogen atom, an oxygen atom, and a sulfur atom. The number of carbon atoms in the above-described aromatic hetero ring is not particularly limited, but is preferably 5 to 18. Specific examples of the above-described aromatic hetero ring include a pyridine ring, a pyridazine ring, a pyrimidine ring, a pyrazine ring, a triazine ring, a thiophene ring, a thiazole ring, and an imidazole ring. The above-described aromatic hetero ring constitutes an aromatic heterocyclic group by removing two hydrogen atoms on the ring.

The aromatic hydrocarbon ring group and aromatic heterocyclic group may have a substituent. The type of the substituent is not particularly limited, and examples thereof include known substituents. Examples thereof include a halogen atom, an alkyl group, an alkoxy group, an aryl group, a hydroxyl group, an amino group, a carboxyl group, a sulfonamide group, an N-sulfonylamide group, an acyl group, an acyloxy group, a cyano group, a nitro group, and an alkoxycarbonyl group. Each of the above-described groups may be further substituted with a substituent. For example, a hydrogen atom in the alkyl group may be replaced with a fluorine atom. In addition, the number of substituents is not particularly limited, and the aromatic hydrocarbon ring group and aromatic heterocyclic group may have one substituent or may have a plurality of substituents.

Among these, as the substituent, from the viewpoint that solubility of the compound represented by General Formula (LC) is further improved, a fluorine atom, a chlorine atom, a fluoroalkyl group, an alkoxy group, or an alkyl group is preferable, and a fluoroalkyl group, an alkoxy group, or an alkyl group is more preferable.

The number of carbon atoms in the fluoroalkyl group and alkyl group, and the number of carbon atoms in an alkyl group of the alkoxy group are not particularly limited, but are preferably 1 to 10, more preferably 1 to 5, still more preferably 1 to 3, and particularly preferably 1.

The fluoroalkyl group is a group in which at least one hydrogen atom in the alkyl group is replaced with a fluorine atom, and it is preferable that all hydrogen atoms are replaced with fluorine atoms (so-called perfluoroalkyl group is preferable).

As A¹¹ to A¹⁵, an aromatic hydrocarbon ring group which may have a substituent is preferable, and a phenylene group bonded at the 1-position and the 4-position is more preferable.

In General Formula (LC), the divalent linking group represented by Z¹¹ to Z¹⁴ is not particularly limited, and examples thereof include a divalent aliphatic hydrocarbon group (which may be linear, branched, or cyclic; a divalent aliphatic hydrocarbon group having 1 to 20 carbon atoms is preferable, and examples thereof include an alkylene group; in addition, the divalent aliphatic hydrocarbon group may be an alkenylene group or an alkynylene group), —O—, —S—, —SO₂—, —NR¹—, —CO— (—C(═O)—), —COO— (—C(═O)O—), —OCO— (—OC(═O)—), —CO—NR′—, —SO₃—, —SO₂NR′—, —NR¹SO₂—, —N═N—, —CH═N—, —N═CH—, and a group of a combination of two or more these groups. Here, R′ represents a hydrogen atom or an alkyl group (preferably having 1 to 10 carbon atoms). A hydrogen atom in the above-described divalent linking group may be replaced with another substituent such as a halogen atom.

Among these, as Z¹¹ to Z¹⁴, —COO—, —OCO—, or —CH═CH— is preferable.

In general Formula (LC), m³ and m⁴ each independently represent an integer of 0 or 1, preferably 0.

The compound represented by General Formula (LC) can be synthesized by a known method.

Specific examples of the above-described compound represented by General Formula (LC) are described below, but the compound is not limited thereto.

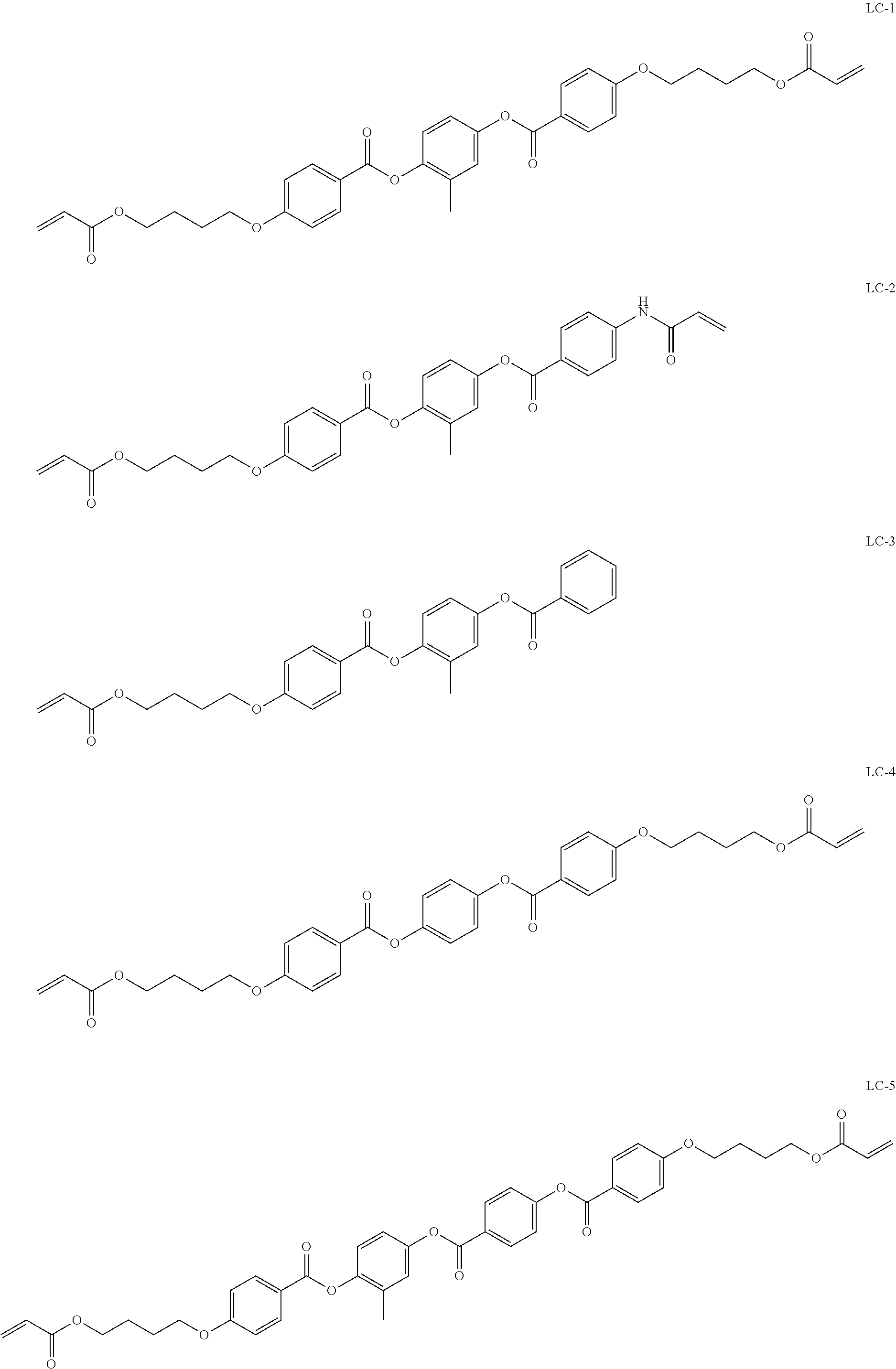

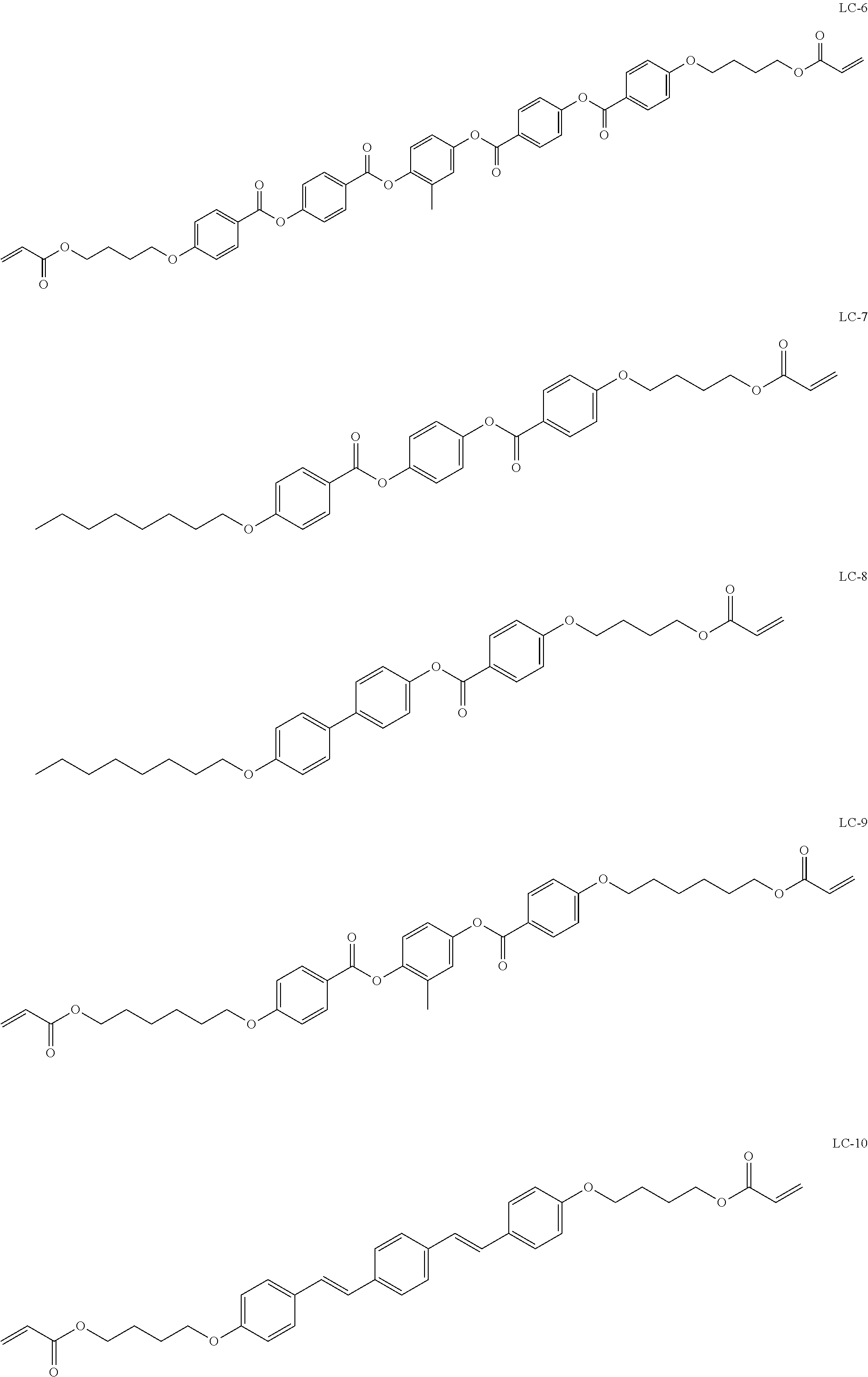

The compound represented by General Formula (LC) may be used alone or in combination of a plurality thereof.

The content of the liquid crystalline compound in the liquid crystal composition according to the embodiment of the present invention is preferably 5% to 99% by mass, more preferably 25% to 98% by mass, still more preferably 65% to 98% by mass, and particularly preferably 70% to 98% by mass with respect to the total mass of the liquid crystal composition.

[Specific Compound or Mixture]

The liquid crystal composition according to the embodiment of the present invention contains a specific compound or a mixture. The specific compound and mixture are as described above. The specific compound may be used alone or in combination of a plurality thereof.

The content of the specific compound or mixture in the liquid crystal composition according to the embodiment of the present invention is preferably 1% to 20% by mass, more preferably 2% to 15% by mass, and still more preferably 2% to 10% by mass with respect to the total mass of the liquid crystalline compound.

[Polymerization Initiator]

The liquid crystal composition may include a polymerization initiator.

The polymerization initiator is preferably a photopolymerization initiator capable of initiating a polymerization reaction by ultraviolet irradiation. Examples of the photopolymerization initiator include an α-carbonyl compound, acyloin ether, an α-hydrocarbon-substituted aromatic acyloin compound, a polynuclear quinone compound, a phenazine compound, and an oxadiazole compound.

The content of the polymerization initiator in the liquid crystal composition is not particularly limited, but is preferably 0.1% to 20% by mass and more preferably 1% to 8% by mass with respect to the total mass of the liquid crystalline compound.

In addition to the above-described components, the liquid crystal composition may also include other additives such as a solvent, an alignment control agent, an antioxidant, an ultraviolet absorber, a sensitizer, a stabilizer, a plasticizer, a chain transfer agent, a polymerization inhibitor, an anti-foaming agent, a leveling agent, a thickener, a flame retardant, a surfactant, a dispersant, a polymerizable monomer, and a coloring material such as a dye and a pigment.

[Uses]

The above-described liquid crystal composition can be applied to various uses. For example, the liquid crystal composition can be used to form a screen, an optically anisotropic body, or a reflective film. For example, in a case where the liquid crystalline compound has a polymerizable group, a cured product can be obtained by subjecting a liquid crystalline composition to a curing treatment (light irradiation treatment, heat treatment, or the like), and the cured product can be suitably applied to an optically anisotropic body or a reflective film.

The optically anisotropic body is intended to be a substance having optical anisotropy.

In addition, the reflective film corresponds to a layer obtained by immobilizing the cholesteric liquid crystalline phase, and can reflect light in a predetermined reflection band. The reflective film can be suitably applied to, for example, a transparent screen.

The method for curing the liquid crystal composition will be described below.

A method for curing (polymerizing and curing) the liquid crystal composition is not particularly limited, and a known method can be adopted. Examples thereof include an aspect which includes a step X of bringing a predetermined substrate into contact with the liquid crystal composition to form a composition layer on the substrate, a step Y of exposing the composition layer, and a step Z of subjecting the composition layer to a curing treatment.

According to this aspect, the liquid crystalline compound can be immobilized in an aligned state, and a so-called optically anisotropic body or a layer obtained by immobilizing a cholesteric liquid crystalline phase can be formed.

Hereinafter, the procedures of steps X to Z will be described in detail.

The step X is a step of bringing a substrate into contact with the liquid crystal composition to form a composition layer on the substrate. The type of the substrate to be used is not particularly limited, and examples thereof include known substrates (for example, a resin substrate, a glass substrate, a ceramic substrate, a semiconductor substrate, and a metal substrate).

A method of bringing the substrate into contact with the liquid crystal composition is not particularly limited, and examples thereof include a method of applying the liquid crystal composition to the substrate and a method of immersing the substrate in the liquid crystal composition.

After bringing the substrate into contact with the liquid crystal composition, as necessary, a drying treatment may be performed in order to remove a solvent from the composition layer on the substrate. In addition, a heat treatment may be performed in order to promote the alignment of the liquid crystalline compound to be a liquid crystalline phase.

The step Y is a step of subjecting the composition layer to an exposure treatment using i-rays or the like.

The above-described specific compound undergoes photoisomerization due to the exposure treatment, so that HTP increases. As a result, the liquid crystalline compound in the composition layer is aligned to form a cholesteric liquid crystalline phase.

In the exposure treatment, the degree of change in HTP can also be adjusted by appropriately adjusting the exposure amount, and/or the exposure wavelength and the like. In addition, after the exposure, a heat treatment may be further performed in order to promote the alignment of the liquid crystalline compound to be a liquid crystalline phase.

The step Z is a step of subjecting the composition layer undergone the step Y to a curing treatment.

A method of the curing treatment is not particularly limited, and examples thereof include a photo-curing treatment and a thermal-curing treatment. Among these, a photo-curing treatment is preferable.

In a case where a photo-curing treatment is performed as the curing treatment, it is preferable that the liquid crystal composition includes a photopolymerization initiator. It is preferable that the wavelength of irradiation light in the photo-curing treatment is different from the wavelength of irradiation light in the above-described exposure treatment.

By the above-described curing treatment, a layer obtained by immobilizing the cholesteric liquid crystalline phase is formed. The layer formed by immobilizing the cholesteric liquid crystalline phase no longer needs to exhibit liquid crystallinity anymore. More specifically, for example, as a state in which the cholesteric liquid crystalline phase is “immobilized,” the most typical and preferred aspect is a state in which the alignment of the liquid crystalline compound, which is the cholesteric liquid crystalline phase, is retained. More specifically, the state is preferably a state in which the layer does not exhibit fluidity within a temperature range of usually 0° C. to 50° C., and under more severe conditions of a temperature range of −30° C. to 70° C., and in which the immobilized alignment morphology can be kept stable without being changed due to an external field or an external force.

### EXAMPLES

Hereinafter, the present invention will be described in more detail based on examples. The materials, the amounts of materials to be used, the proportions, the treatment details, the treatment procedure, or the like shown in the examples below may be modified appropriately as long as the modifications do not depart from the spirit of the present invention. Accordingly, the scope of the present invention should not be construed as being limited to the following examples.

### Example 1

### Synthesis of Compound CD-1

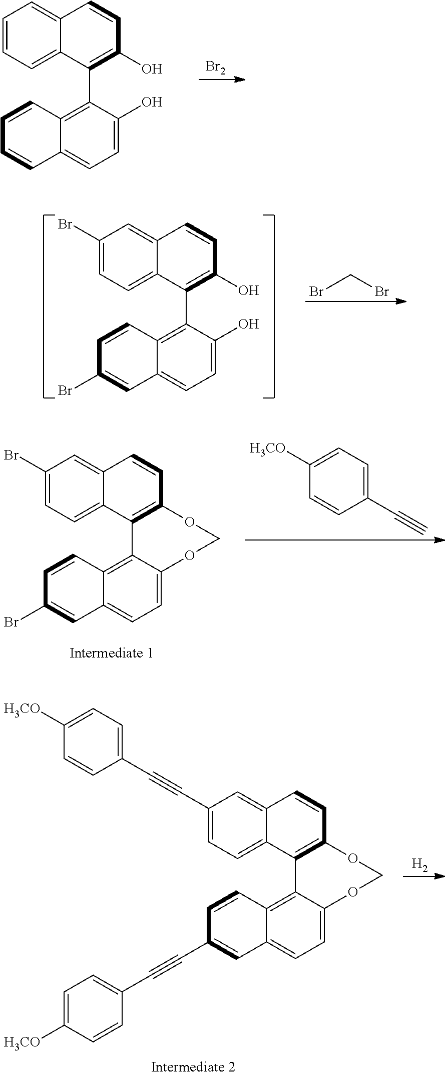

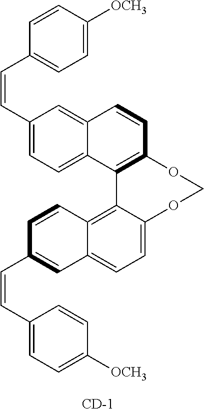

### Synthesis of Intermediate 1

65.0 g of (R)-binaphthol (manufactured by KANTO CHEMICAL CO., INC.) and 500 mL of butyl acetate (manufactured by Wako Pure Chemical Industries, Ltd.) were placed in a 2 L three-neck flask, 100 g of bromine (manufactured by Wako Pure Chemical Industries, Ltd.) was added dropwise thereto at 0° C., and the mixture was stirred for 5 hours. Subsequently, the obtained reaction solution was washed with sodium hydrogen sulfite water (21.7 g of sodium hydrogen sulfite (manufactured by Wako Pure Chemical Industries, Ltd.) and 290 mL of water), 325 mL of water, and sodium hydrogen carbonate water (13.0 g of sodium hydrogen carbonate (manufactured by Wako Pure Chemical Industries, Ltd.) and 300 mL of water) in this order. The washed solution was dried over magnesium sulfate, the solvent was evaporated from the solution under reduced pressure, and the residue was transferred to a three-neck flask.

Subsequently, 80.2 g of N,N-dimethylformamide (DMF, manufactured by Wako Pure Chemical Industries, Ltd.), 78.0 g of potassium carbonate (manufactured by Wako Pure Chemical Industries, Ltd.), 75.0 g of butyl acetate (manufactured by Wako Pure Chemical Industries, Ltd.), and 43.5 g of dibromomethane (manufactured by Wako Pure Chemical Industries, Ltd.) were added to the above-described three-neck flask, and the mixture was stirred at 90° C. for 4 hours. The obtained reaction solution was cooled to room temperature, and the solid was filtered off. 170 mL of ethyl acetate (manufactured by Wako Pure Chemical Industries, Ltd.) and 550 mL of methanol (manufactured by Wako Pure Chemical Industries, Ltd.) was added to the solution after the solid was filtered off, and the resulting solid was collected by filtration. Next, the obtained solid was blast-dried at 40° C. for 12 hours, thereby obtaining an intermediate 1 (66.0 g, yield: 75%).

### Synthesis of Intermediate 2

20.0 g of the intermediate 1, 17.4 g of ethynyl anisole (manufactured by Tokyo Chemical Industry Co., Ltd.), 0.08 g of copper iodide (manufactured by Wako Pure Chemical Industries, Ltd.), 0.22 g of triphenylphosphine palladium dichloride (manufactured by Tokyo Chemical Industry Co., Ltd.), 120 mL of triethylamine (manufactured by Wako Pure Chemical Industries, Ltd.), and 40 mL of pyridine (manufactured by Wako Pure Chemical Industries, Ltd.) were placed in a 500 mL three-neck flask, and the mixture was stirred at 90° C. for 3 hours. Subsequently, the obtained reaction solution was cooled to 0° C., and 400 mL of methanol (manufactured by Wako Pure Chemical Industries, Ltd.) was added thereto, and the resulting solid was collected by filtration. Next, the obtained solid was blast-dried at 40° C. for 12 hours, thereby obtaining an intermediate 2 (22.0 g, yield: 90%).

### Synthesis of Compound CD-1

20.0 g of the intermediate 2, 10.0 g of Lindlar's catalyst (manufactured by Tokyo Chemical Industry Co., Ltd.), 9.2 g of quinoline (manufactured by Wako Pure Chemical Industries, Ltd.), and 100 mL of 1,4-dioxane (manufactured by Wako Pure Chemical Industries, Ltd.) were placed in a 300 mL three-neck flask, and the mixture was replaced with hydrogen and stirred at 80° C. for 6 hours. The solid was filtered off by Celite filtration, and the obtained solution was purified by column chromatography and blast-dried at 40° C. for 12 hours, thereby obtaining a compound CD-1 (18.0 g, yield: 90%).

¹H NMR of compound CD-1 (deuterated solvent: DMSO (dimethyl sulfoxide)-d₆): δ 8.01 (2H, d), 7.94 (2H, s), 7.52 (2H, d), 7.16 (8H, m), 6.76 (4H, d), 6.67 (4H, d), 5.70 (2H, s), 3.57 (6H, s)

From the measurement result of ¹H NMR of the compound CD-1, it was confirmed that the compound CD-1 included the above-described monovalent substituent represented by General Formula (2) and did not include the above-described monovalent substituent represented by General Formula (4).

[Evaluation of Initial HTP, HTP after Exposure, and Rate of Increase in HTP Due to Exposure]

Using the compound CD-1, the initial (unexposed) helical twisting power (initial HTP), the helical twisting power after exposure (HTP after exposure), and the rate of increase in HTP due to exposure were evaluated.

<Preparation of Sample Solution>

A liquid crystalline compound LC-1 represented by the following structure and the compound CD-1 (chiral compound) were mixed with each other, and a solvent was added to the obtained mixture to prepare a sample solution having the following composition.

| | |
|---|---|
| Liquid crystalline compound LC-1 represented by the | 100 parts by mass |
| following structure | |
| Compound CD-1 | 5 parts by mass |
| Solvent (methyl ethyl ketone (MEK)/cyclohexanone = | amount at which the concentration |
| 90/10 (mass ratio)) | of solute is 30% by mass |

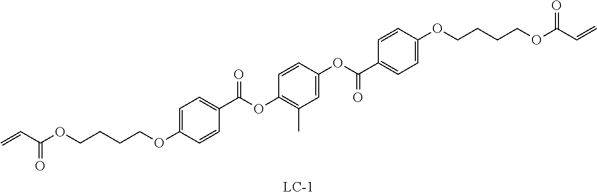 [SMILES: C=CC(=O)OCCCCOc1ccc(C(=O)Oc2ccc(OC(=O)c3ccc(OCCCCOC(=O)C=C)cc3)c(C)c2)cc1]

<Production of Liquid Crystal Layer 1>

Next, a polyimide alignment film SE-130 (manufactured by Nissan Chemical Corporation) was applied to a washed glass substrate to form a coating film. After firing the obtained coating film, the coating film was subjected to a rubbing treatment to produce a substrate with an alignment film. 30 μL of the above-described sample solution was spin-coated on the rubbing-treated surface of the alignment film under the conditions of a rotation speed of 1000 rpm and 10 seconds, and then the substrate was aged at 90° C. for 1 minute to obtain a liquid crystal layer 1.

<Evaluation>

(Initial HTP)

The central reflection wavelength of the liquid crystal layer 1 was measured, and the initial HTP was obtained by Expression (1).

HTP [μm⁻¹]=(average refractive index of liquid crystalline compound)/{(concentration (% by mass) of chiral compound with respect to liquid crystalline compound)×(central reflection wavelength (nm))} Expression (1):

In Expression (1), HTP was calculated on the assumption that the “average refractive index of liquid crystalline compound” was 1.55. In addition, “central reflection wavelength” was measured using a spectroscope (UV-3100 manufactured by Shimadzu Corporation).

(HTP after Exposure)

After irradiating the liquid crystal layer 1 with light having a wavelength of 365 nm at an exposure amount of 100 mJ/cm², the central reflection wavelength of the liquid crystal layer 1 after exposure was measured. Next, the HTP after exposure was obtained by Expression (1) described above.

(Rate of Increase in HTP)

From each of the obtained initial HTP and HTP after exposure values, the rate of increase in HTP was calculated by Expression (2).

Rate of increase in HTP [%]={(HTP after exposure)−(HTP before exposure)}/(HTP before exposure)×100 Expression (2):

Based on the following evaluation standards, the initial HTP, HTP after exposure, and rate of increase in HTP due to exposure were evaluated. The results are shown in Table 1.

<<Evaluation Standard (Initial HTP)>>

“A”: initial HTP was 50 [μm⁻¹] or more.

“B”: initial HTP was 25 [μm⁻¹] or more and less than 50 [μm⁻¹].

“C”: initial HTP was less than 25 [μm⁻¹].

<<Evaluation Standard (HTP after Exposure)>>

“AA”: HTP after exposure was 70 [μm⁻¹] or more.

“A”: HTP after exposure was 50 [μm⁻¹] or more and less than 70 [μm⁻¹].

“B”: HTP after exposure was 30 [μm⁻¹] or more and less than 50 [μm⁻¹].

“C”: HTP after exposure was less than 30 [μm⁻¹].

<<Evaluation Standard (Rate of Increase in HTP)>>

“AA”: rate of increase in HTP was 200% or more.

“A”: rate of increase in HTP was 150% or more and less than 200%.

“B”: rate of increase in HTP was 100% or more and less than 150%.

“C”: rate of increase in HTP was less than 100%.

“D”: HTP did not increase.

[Evaluation of Temperature Dependence of HTP]

By Expression (1) described above, each HTP of the liquid crystal layer 1 (unexposed) at 40° C. and 90° C. was calculated.

Regarding “central reflection wavelength at each temperature (40° C. and 90° C.)”, the central reflection wavelength was measured in a state in which the produced liquid crystal layer was respectively heated to 40° C. and 90° C. using a hot stage (manufactured by METTLER TOLEDO, FP90/FP82HT), using a microscope (manufactured by Nikon Corporation, ECLIPSE E600-POL) and a spectrophotometer (manufactured by Ocean Optics, Inc., USB-4000/USB4H09800).

(Calculation of Rate of Temperature Change in HTP)

The temperature dependence of HTP was evaluated by calculating the rate of temperature change in HTP according to Expression (3).

Rate of temperature change [%]={(HTP at 40° C.)−(HTP at 90° C.)}/(HTP at 40° C.)×100 Expression (3):

From the value calculated from Expression (3), the temperature dependence of HTP was evaluated based on the following evaluation standard. The results are shown in Table 1.

<<Evaluation Standard (Temperature Dependence of HTP)>>

“A”: rate of temperature change was less than 2%.

“B”: rate of temperature change was 2% or more and less than 5%.

“C”: rate of temperature change was 5% or more.

### Example 2

A compound CD-2 was synthesized by the same method as the compound CD-1 described above. The method for synthesizing the compound CD-2 is shown below.

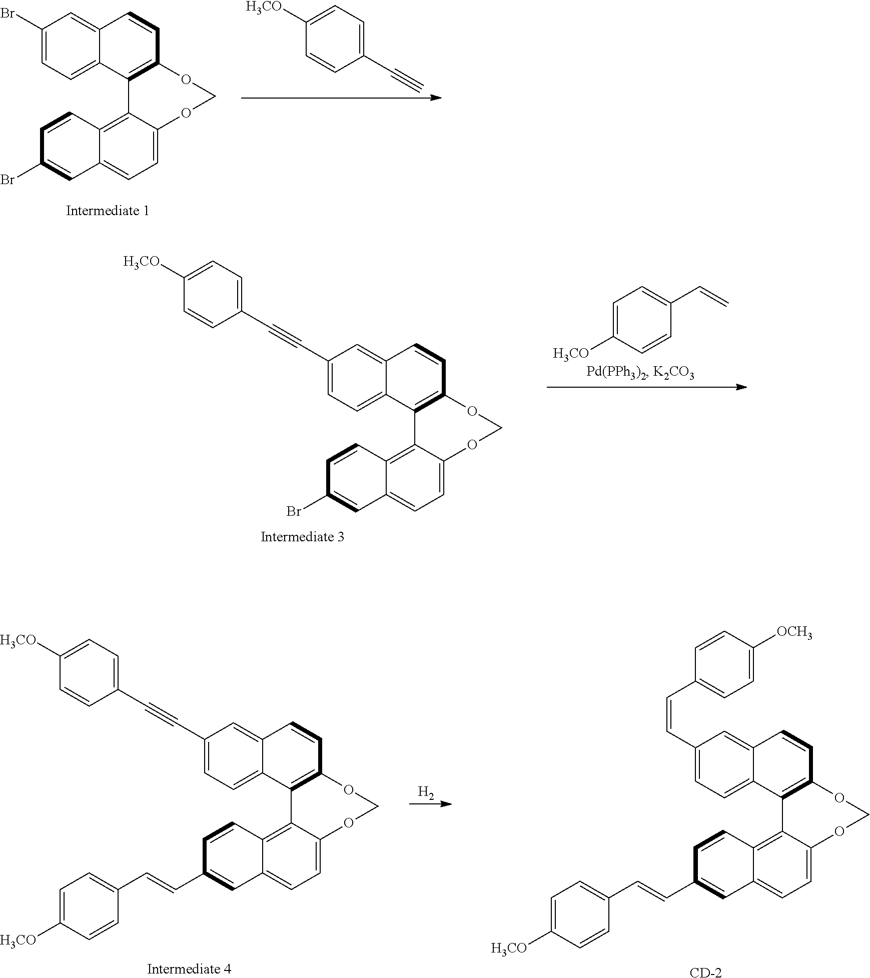

From the measurement result of ¹H NMR (DMSO-d6) of the compound CD-2, it was confirmed that the compound CD-2 included both of the above-described monovalent substituent represented by General Formula (2) and the above-described monovalent substituent represented by General Formula (4), and the content ratio (X1/Y1) of the content (X1 mol) of the monovalent substituent represented by General Formula (2) and the content (Y1 mol) of the monovalent substituent represented by General Formula (4) was 1.

Next, using the above-described compound CD-2, the same evaluations as in Example 1 were performed. The results are shown in Table 1.

### Examples 3 to 13 and Comparative Examples 1 and 2

Compounds CD-3 to CD-13 and comparative compounds CCD-1 and CCD-2 were synthesized in the same manner as in the compound CD-1 described above. Next, using each of the above-described compounds, the same evaluations as in Example 1 were performed. The results are shown in Table 1.

### Example 14

### Synthesis of Compound CD-14

A compound CD-14 was synthesized by the same method as the compound CD-1 described above and a method described in Advanced Synthesis and catalysis, 356, pp. 179 to 188 (2014). Next, using the above-described compound CD-14, the same evaluations as in Example 1 were performed. The results are shown in Table 1.

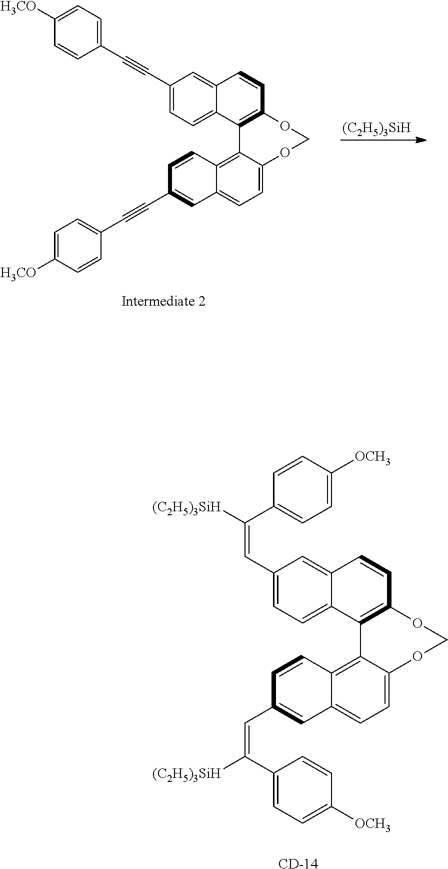

### Example 15

### Synthesis of Compound CD-15

A compound CD-15 was synthesized by the same method as the compound CD-1 described above, a method described in Tetrahedron, 72, pp. 1553 to 1540 (2016), and a method described in Organic Chemistry, 9, pp. 1883 to 1890 (2013). Next, using the above-described compound CD-15, the same evaluations as in Example 1 were performed. The results are shown in Table 1.

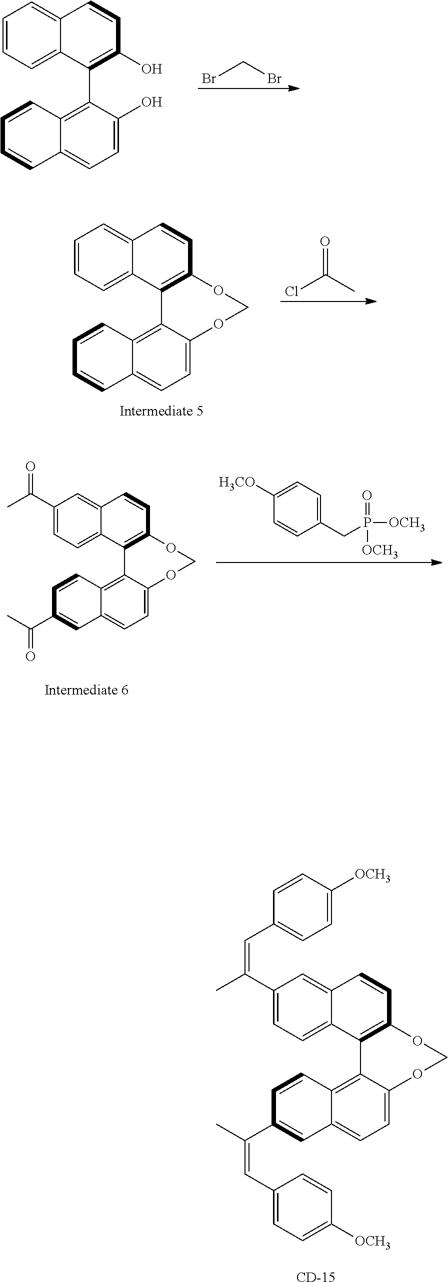

### Examples 16 to 18

### Synthesis of compound CCD-3

A compound CCD-3 was synthesized by the same method as the compound CD-1 and compound CD-2 described above.

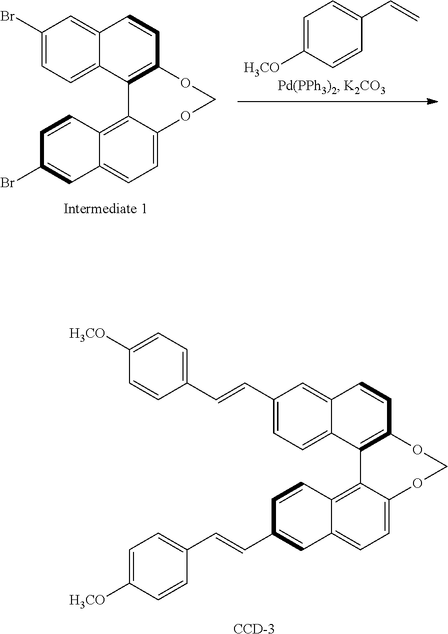

From the measurement result of ¹H NMR (DMSO-d6) of the compound CCD-3, it was confirmed that the compound CCD-3 did not include the above-described monovalent substituent represented by General Formula (2) and included the above-described monovalent substituent represented by General Formula (6). The compound CCD-3 corresponds to the above-described compound represented by General Formula (Y1).

### Example 16

### Preparation of Sample Solution

A mixture was prepared by mixing the above-described compound CD-1 and compound CD-2 at the mixing ratio shown in Table 1. Next, the above-described liquid crystalline compound LC-1 was mixed with the above-described mixture consisting of two kinds of chiral compounds, and a solvent was added to the obtained mixture to prepare a sample solution having the following composition.

| | |
|---|---|
| Liquid crystalline compound LC-1 | 100 parts by mass |
| Mixture of compound CD-1 and compound CD-2 | 5 parts by mass |
| Solvent (methyl ethyl ketone (MEK)/ | amount at which the |
| cyclohexanone = 90/10 (mass ratio)) | concentration of |
| | solute is 30% by mass |

Next, a liquid crystal layer was produced by the same method as the method for producing the liquid crystal layer 1 of Example 1, except that the above-described sample solution was used. In addition, the evaluations were performed by the same method as in Example 1. The results are shown in Table 2.

### Example 17

A liquid crystal layer was produced by the same method as in Example 16, except that the above-described compound CD-1 and compound CD-2 were mixed at the mixing ratio shown in Table 1. In addition, the evaluations were performed by the same method as in Example 1. The results are shown in Table 2.

### Example 18

A liquid crystal layer was produced by the same method as in Example 16, except that the above-described compound CD-1 and compound CCD-3 were mixed at the mixing ratio shown in Table 1. In addition, the evaluations were performed by the same method as in Example 1. The results are shown in Table 2.

Tables 1 and 2 are shown below.

In Table 1, “Content ratio X1/Y1” is a content ratio of, in the compounds, the content (X1 mol) of the monovalent substituent represented by General Formula (2) and the content (Y1 mol) of the monovalent substituent represented by General Formula (4), and is intended to be a value obtained by dividing X1 by Y1.

In Table 1, the compound CD-1 and compounds CD-3 to CD-15 correspond to General Formula (X1) described above.

In Table 2, “Content ratio X2/Y2” is a content ratio of, in the mixtures, the content (X2 mol) of the monovalent substituent represented by General Formula (2) to the content (Y2 mol) of the monovalent substituent represented by General Formula (6), and is intended to be a value obtained by dividing X2 by Y2.

In Table 2, the compound CD-1 and compound CD-2 are intended to be the compound CD-1 used in Example 1 and the compound CD-2 used in Example 2.

In Table 2, the compound CD-2 and compound CCD-3 correspond to General Formula (Y1) described above.

TABLE 1

| Chiral compound | | | | | | | | | | | |
|---|---|---|---|---|---|---|---|---|---|---|---|
| | 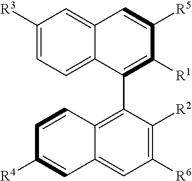 | | | | | | | | | | |
| | | | | | | | | Evaluation result | | | |
| | | | | | | | | | | | Temperature |
| | | | | | | | Content | Initial | HTP | Rate of | dependence |
| Type | R¹ | R² | R³ | R⁴ | R⁵ | R⁶ | ratio X1/Y1 | HTP | after exposure | increase in HTP | of HTP |

| | | | | | | | | | | | |
|---|---|---|---|---|---|---|---|---|---|---|---|
| Example 1 | CD-1 | 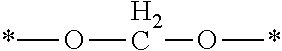 [SMILES: *OCO*] | 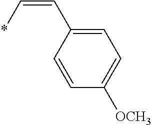 [SMILES: */C=C\c1ccc(OC)cc1] | 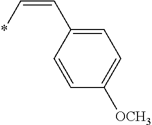 [SMILES: */C=C\c1ccc(OC)cc1] | H | H | Only X1 | C | A | AA | A |
| Example 2 | CD-2 | 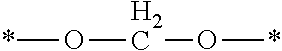 [SMILES: *OCO*] | 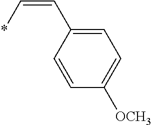 [SMILES: */C=C\c1ccc(OC)cc1] | 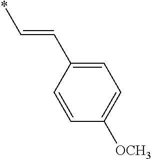 [SMILES: */C=C/c1ccc(OC)cc1] | H | H | 1 | A | A | C | A |
| Example 3 | CD-3 | 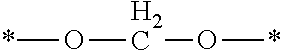 [SMILES: *OCO*] | 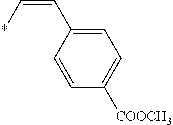 [SMILES: */C=C\c1ccc(C(=O)OC)cc1] | 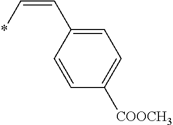 [SMILES: */C=C\c1ccc(C(=O)OC)cc1] | H | H | Only X1 | C | A | AA | A |
| Example 4 | CD-4 | 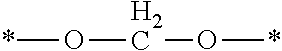 [SMILES: *OCO*] | 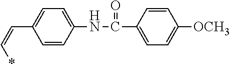 [SMILES: */C=C\c1ccc(NC(=O)c2ccc(OC)cc2)cc1] | 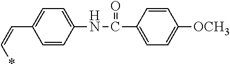 [SMILES: */C=C\c1ccc(NC(=O)c2ccc(OC)cc2)cc1] | H | H | Only X1 | C | AA | AA | A |
| Example 5 | CD-5 | 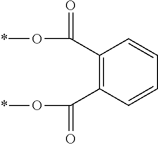 [SMILES: *OC(=O)c1ccccc1C(=O)O*] | 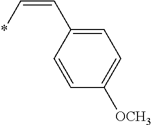 [SMILES: */C=C\c1ccc(OC)cc1] | 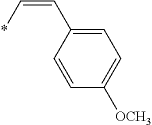 [SMILES: */C=C\c1ccc(OC)cc1] | H | H | Only X1 | C | AA | AA | A |
| Example 6 | CD-6 | 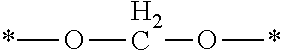 [SMILES: *OCO*] | 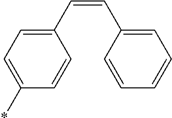 [SMILES: *c1ccc(/C=C\c2ccccc2)cc1] | 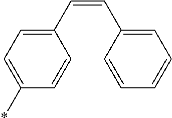 [SMILES: *c1ccc(/C=C\c2ccccc2)cc1] | H | H | Only X1 | B | AA | B | A |
| Example 7 | CD-7 | 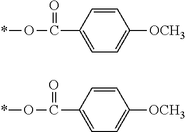 [SMILES: *OC(=O)c1ccc(OC)cc1 ; *OC(=O)c1ccc(OC)cc1] | 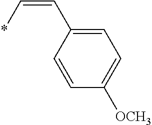 [SMILES: */C=C\c1ccc(OC)cc1] | 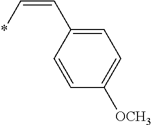 [SMILES: */C=C\c1ccc(OC)cc1] | H | H | Only X1 | C | B | A | C |

TABLE 2

| | Chiral compound | | | | | | | | | | | |
|---|---|---|---|---|---|---|---|---|---|---|---|---|
| | | 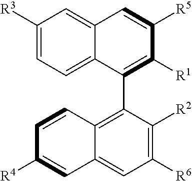 | | | | | | | | | | |
| | | | | | | | | | Evaluation result | | | |
| | | | | | | | | Content | | HTP | Rate of | Temperature |
| | | | | | | | | ratio | Initial | after | increase | dependence |
| | Type | R¹ | R² | R³ | R⁴ | R⁵ | R⁶ | X1/Y1 | HTP | exposure | in HTP | of HTP |
| Example 8 | CD-8 | OCH₃ | OCH₃ | 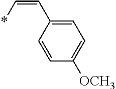 [SMILES: */C=C\c1ccc(OC)cc1] | 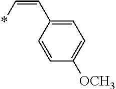 [SMILES: */C=C\c1ccc(OC)cc1] | H | H | Only X1 | C | C | AA | C |
| Example 9 | CD-9 | 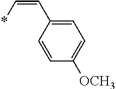 [SMILES: */C=C\c1ccc(OC)cc1] | 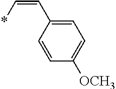 [SMILES: */C=C\c1ccc(OC)cc1] | H | H | H | H | Only X1 | C | C | A | B |
| Example 10 | CD-10 | 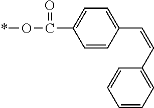 [SMILES: *OC(=O)c1ccc(/C=C\c2ccccc2)cc1] | 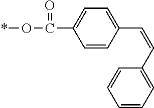 [SMILES: *OC(=O)c1ccc(/C=C\c2ccccc2)cc1] | H | H | H | | Only X1 | C | C | C | C |

| | | | | | | | | | | | |
|---|---|---|---|---|---|---|---|---|---|---|---|
| Example 11 | CD-11 | 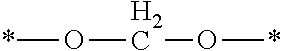 [SMILES: *OCO*] | 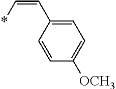 [SMILES: */C=C\c1ccc(OC)cc1] | H | H | H | Only X1 | C | C | C | A |
| Example 12 | CD-12 | 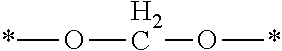 [SMILES: *OCO*] | H | H | 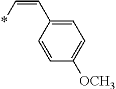 [SMILES: */C=C\c1ccc(OC)cc1] | 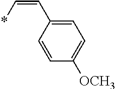 [SMILES: */C=C\c1ccc(OC)cc1] | Only X1 | C | A | AA | A |
| Example 13 | CD-13 | 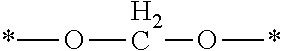 [SMILES: *OCO*] | 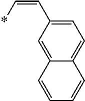 [SMILES: */C=C\c1ccc2ccccc2c1] | 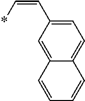 [SMILES: */C=C\c1ccc2ccccc2c1] | H | H | Only X1 | C | A | B | A |

TABLE 3

| | Chiral compound | | | | | | | | | | | |
|---|---|---|---|---|---|---|---|---|---|---|---|---|
| | | 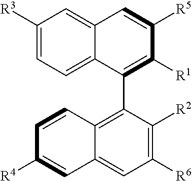 | | | | | | | | | | |
| | | | | | | | | | Evaluation result | | | |
| | | | | | | | | Content | | HTP | Rate of | Temperature |
| | | | | | | | | ratio | Initial | after | increase | dependence |
| | Type | R¹ | R² | R³ | R⁴ | R⁵ | R⁶ | X1/Y1 | HTP | exposure | in HTP | of HTP |
| Example 14 | CD-14 | 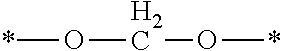 [SMILES: *OCO*] | | 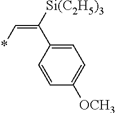 | 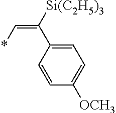 | H | H | Only Y1 | C | C | C | A |
| Example 15 | CD-15 | 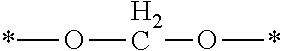 [SMILES: *OCO*] | | 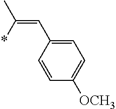 [SMILES: */C(C)=C\c1ccc(OC)cc1] | 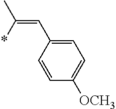 [SMILES: */C(C)=C\c1ccc(OC)cc1] | H | H | Only Y1 | C | C | C | A |
| Comparative Example 1 | CCD-1 | 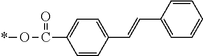 [SMILES: *OC(=O)c1ccc(/C=C/c2ccccc2)cc1] | 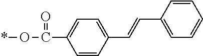 [SMILES: *OC(=O)c1ccc(/C=C/c2ccccc2)cc1] | H | H | H | H | Only Y1 | A | C | D | C |
| Comparative Example 2 | CCD-2 | OH | OH | *—CH═CH—COOC₂H₅ | *—CH═CH—COOC₂H₅ | H | H | Only Y1 | B | C | D | C |

TABLE 4

| | Mixture | | Evaluation result | | | |
|---|---|---|---|---|---|---|
| | Com- | | | HTP | Rate of | Tem- |
| | position | Content | | after | in- | perature |
| | (molar | ratio | Initial | ex- | crease | dependence |
| | ratio) | X2/Y2 | HTP | posure | in HTP | of HTP |
| Example | CD-1/ | 5 | B | A | B | A |
| 16 | CD-2 = | | | | | |
| | 2/1 | | | | | |
| Example | CD-1/ | 10 | C | A | A | A |
| 17 | CD-2 = | | | | | |
| | 9/2 | | | | | |
| Example | CD-1/ | 1 | A | A | C | A |
| 18 | CCD-3 = | | | | | |
| | 1/1 | | | | | |

From the results in Table 1, it was confirmed that, in the compounds of Examples, the intensity of HTP was increased by exposure to irradiation with light such as ultraviolet rays and the rate of increase in HTP thereof was also excellent.

From the comparison of Example 1 with Example 2, it was confirmed that, in a case where the specific compound did not include a substituent represented by General Formula (4), the rate of increase in HTP was large.

In addition, from the comparison of Example 1 with Example 6, it was confirmed that, in a case where L¹ in the specific compound was a single bond, the rate of increase in HTP was large.

In addition, from the comparison of Example 1 with Examples 7 and 8, it was confirmed that, in a case where R¹ and R² in the specific compound were bonded to each other to form a ring structure, HTP after exposure was large and the temperature dependence of HTP was small. In addition, from the comparison of Example 1 with Examples 7 to 10, it was also confirmed that, in a case where R¹ and R² in the specific compound were bonded to each other to form a ring structure, HTP after exposure was large and the temperature dependence of HTP was small.

In addition, from the comparison of Example 1 with Example 11, it was confirmed that, in a case where, in the specific compound, one or more groups selected from the group consisting of R¹, R³, and R⁵ represented the monovalent substituent represented by General Formula (2) and one or more groups selected from the group consisting of R², R⁴, and R⁶ represented the monovalent substituent represented by General Formula (2), HTP after exposure and the rate of increase in HTP were large.

In addition, from the comparison of Example 1 with Examples 4 to 6, it was found that the specific compound included a structure in which three or more aromatic hydrocarbon ring groups were linked to each other through a single bond or a divalent linking group (however, the linking of naphthalene rings in the binaphthyl skeleton was not included), HTP after exposure was large.

In addition, from the comparison of Example 1 with Example 13, it was confirmed that, in a case where Ar¹ in General Formula (2) was a benzene ring, the rate of increase in HTP was large.

In addition, from the comparison of Example 1 with Examples 14 and 15, it was confirmed that, in a case where R⁷ and R⁸ in General Formula (2) were hydrogen atoms, HTP after exposure and the rate of increase in HTP were large.

Furthermore, from the comparison of Examples 16 to 18 in Table 2, it was confirmed that, in a case where the value of X2/Y2 in the mixture was 5 or more (preferably 10 or more), the rate of increase in HTP was large.

On the other hand, it was also found that, as the value of X2/Y2 in the mixture was smaller, HTP of the mixture before exposure was larger. Therefore, in a case where a specific compound including no substituent represented by General Formula (4) (corresponding to the above-described compound represented by General Formula (X1)) is mixed with the compound represented by General Formula (Y1) at an arbitrary proportion, a desired initial HTP and rate of increase in HTP can be set in the obtained mixture.

### [Example 19: Production of Reflective Film] [Preparation of Liquid Crystal Composition]

A liquid crystal composition was prepared with the formulation shown below.

| | |
|---|---|
| Liquid crystalline compound LC-1 described | 100 parts by mass |
| above | |
| Compound CD-1 | 5 parts by mass |
| Surfactant S-1 shown below | 0.1 parts by mass |
| IRGACURE 907 (manufactured by BASF) | 3 parts by mass |
| Solvent (methyl ethyl ketone (MEK)/ | amount at which the |
| cyclohexanone = 90/10 (mass ratio)) | concentration of solute |
| | is 30% by mass |

The surfactant S-1 is a compound described in JP5774518B, and has the following structure.

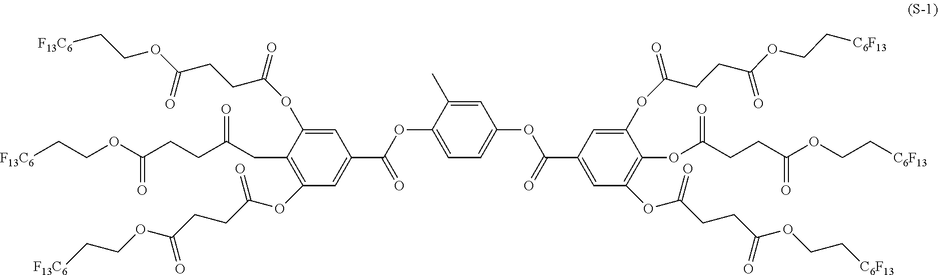

[Production of Reflective Film]

A polyimide alignment film material SE-130 (manufactured by Nissan Chemical Corporation) was applied to a washed glass substrate to form a coating film. After firing the obtained coating film, the coating film was subjected to a rubbing treatment to produce a substrate with an alignment film. 40 μL of the above-described liquid crystal composition was spin-coated on the rubbing-treated surface of the alignment film under the conditions of a rotation speed of 1500 rpm for 10 seconds to form a composition layer. Thereafter, the composition layer was dried (aged) at 90° C. for 1 minute, thereby aligning the liquid crystalline compound in the composition layer (in other words, obtaining a composition layer in a state of the cholesteric liquid crystalline phase).

Next, the composition layer in which the liquid crystalline compound had been aligned was irradiated with light, which is emitted from a light source (2UV Transilluminator manufactured by UVP Inc.) and has a wavelength of 365 nm, at an irradiation intensity of 3.0 mW/cm² for 30 seconds through a mask having an opening portion (corresponding to the treatment of increasing HTP of CD-1). Due to the difference between the opening portion and the non-opening portion of the mask, the composition layer was in a state of having a portion irradiated with light having a wavelength of 365 nm and a portion not irradiated with light.

Subsequently, in a state of removing the mask, the composition layer was subjected to a curing treatment by irradiation with ultraviolet rays (manufactured by HOYA-SCHOTT CORPORATION, EXECURE 3000-W) at an irradiation amount of 500 mJ/cm² under a nitrogen atmosphere at 25° C., thereby obtaining a reflective film (corresponding to a layer obtained by immobilizing the cholesteric liquid crystalline phase).

In the obtained reflective film, it was found that the portion irradiated with light having a wavelength of 365 nm exhibited a short-wavelength reflection and the portion not irradiated exhibited a long-wavelength reflection, and that the selective reflection wavelengths were different (that the helical pitches of the cholesteric layer were different).

What is claimed is:

1. A compound represented by General Formula (1), 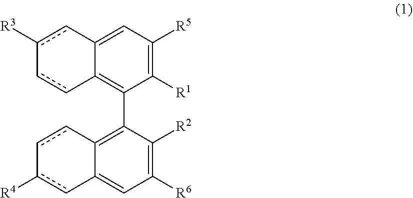
in General Formula (1), R¹ to R⁶ each independently represent a hydrogen atom or a monovalent substituent, where at least one of R¹, R², R³, R⁴, R⁵, or R⁶ represents a monovalent substituent represented by General Formula (2), a portion where a solid line and a broken line are parallel to each other represents a single bond or a double bond, and R¹ and R² may be bonded to each other to form a ring structure; 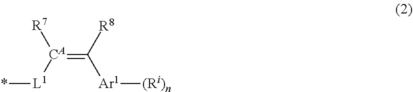
in General Formula (2), Ar¹ represents an (n+1)-valent aromatic hydrocarbon ring group, C^{A} represents a carbon atom, R⁷ and R⁸ each independently represent a hydrogen atom, a cyano group, a substituted boryl group, a substituted silyl group, a substituted aluminum group, a halogen atom, an alkoxycarbonyl group, an alkylcarbonyl group, or a monovalent aliphatic hydrocarbon group having 1 to 10 carbon atoms, Rⁱ represents a monovalent substituent, n represents an integer of 0 to 5, L¹ represents a single bond or a divalent linking group represented by General Formula (3), and * represents a bonding position to a binaphthyl skeleton in General Formula (1), where, in a case where n is 2 or more, a plurality of Rⁱ's may be the same or different from each other;
*-L²-Ar²—** (3)
in General Formula (3), L² represents a single bond or a divalent linking group, Are represents a divalent aromatic hydrocarbon ring group, * represents a bonding position to the binaphthyl skeleton in General Formula (1), and ** represents a bonding site to C^{A} in General Formula (2).

2. The compound according to claim 1,
wherein L¹ represents a single bond.

3. The compound according to claim 1,
wherein one or more groups selected from the group consisting of R¹, R³, and R⁵ represent the monovalent substituent represented by General Formula (2), and
one or more groups selected from the group consisting of R², R⁴, and R⁶ represent the monovalent substituent represented by General Formula (2).

4. The compound according to claim 1,
wherein R¹ and R² are bonded to each other to form a ring structure.

5. The compound according to claim 1,
wherein L¹ represents the divalent linking group represented by General Formula (3),
Rⁱ represents *-L^{S1}-aromatic hydrocarbon ring group, or
R¹ and R² are bonded to each other to represent *-L^{S2}-divalent aromatic hydrocarbon ring group-L^{S2}-*,
where, L^{S1} and L^{S2} each independently represent a single bond or a divalent linking group, and * represents a bonding position.

6. The compound according to claim 1,
wherein R⁷ and R⁸ represent hydrogen atoms.

7. The compound according to claim 1,
wherein Ar¹ represents a benzene ring group.

8. The compound according to claim 1,
wherein all of R¹ to R⁶ represent a monovalent substituent other than a monovalent substituent represented by General Formula (4), 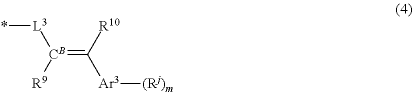
in General Formula (4), Ar³ represents an (m+1)-valent aromatic hydrocarbon ring group, C^{B} represents a carbon atom, R⁹ and R¹⁰ each independently represent a hydrogen atom, a cyano group, a substituted boryl group, a substituted silyl group, a substituted aluminum group, a halogen atom, an alkoxycarbonyl group, an alkylcarbonyl group, or a monovalent aliphatic hydrocarbon group having 1 to 10 carbon atoms, EU represents a monovalent substituent, m represents an integer of 0 to 5, L³ represents a single bond or a divalent linking group represented by General Formula (5), and * represents a bonding position to the binaphthyl skeleton in General Formula (1),
where, in a case where m is 2 or more, a plurality of 1V's may be the same or different from each other;
*-L⁴-Ar⁴-** (5)
in General Formula (5), Ar⁴ represents a divalent aromatic hydrocarbon ring group, L⁴ represents a single bond or a divalent linking group, * represents a bonding position to the binaphthyl skeleton in General Formula (1), and ** represents a bonding site to C^{B} in General Formula (4).

9. A mixture consisting of the compound according to claim 8 and a compound represented by General Formula (Y1), 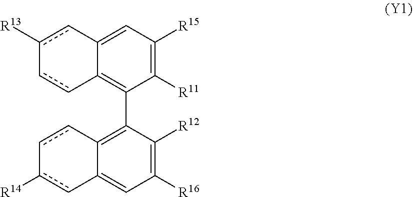
in General Formula (Y1), R¹¹ to R¹⁶ each independently represent a hydrogen atom or a monovalent substituent, where at least R¹¹, R¹², R¹³, R¹⁴, R¹⁵, or R¹⁶ represents a monovalent substituent represented by General Formula (6), a portion where a solid line and a broken line are parallel to each other represents a single bond or a double bond, and R¹¹ and R¹² may be bonded to each other to form a ring structure; 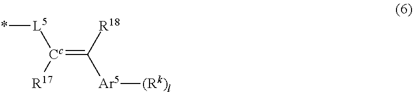
in General Formula (6), Ar⁵ represents an (l+1)-valent aromatic hydrocarbon ring group, C^{C} represents a carbon atom, R¹⁷ and R¹⁸ each independently represent a hydrogen atom, a cyano group, a substituted boryl group, a substituted silyl group, a substituted aluminum group, a halogen atom, an alkoxycarbonyl group, an alkylcarbonyl group, or a monovalent aliphatic hydrocarbon group having 1 to 10 carbon atoms, R^{k} represents a monovalent substituent, 1 represents an integer of 0 to 5, L⁵ represents a single bond or a divalent linking group represented by General Formula (7), and * represents a bonding position to a binaphthyl skeleton in General Formula (Y1),
where, in a case where 1 is 2 or more, a plurality of R^{k}'s may be the same or different from each other;
*-L⁶-Ar⁶-** (7)
in General Formula (7), Ar⁶ represents a divalent aromatic hydrocarbon ring group,
L⁶ represents a single bond or a divalent linking group, * represents a bonding position to the binaphthyl skeleton in General Formula (Y1), ** represents a bonding site to C^{C} in General Formula (6).

10. The mixture according to claim 9,
wherein a ratio of a content of the monovalent substituent represented by General Formula (2) to a content of the monovalent substituent represented by General Formula (6) is 5 or more.

11. A liquid crystal composition comprising:
a liquid crystalline compound; and
the compound according to claim 1.

12. A cured product obtained by curing the liquid crystal composition according to claim 11.

13. An optically anisotropic body formed by using the liquid crystal composition according to claim 11.

14. A reflective film formed by using the liquid crystal composition according to claim 11.

15. A liquid crystal composition comprising:
a liquid crystalline compound; and
the mixture according to claim 9.

16. The compound according to claim 2,
wherein one or more groups selected from the group consisting of R¹, R³, and R⁵ represent the monovalent substituent represented by General Formula (2), and
one or more groups selected from the group consisting of R², R⁴, and R⁶ represent the monovalent substituent represented by General Formula (2).

17. The compound according to claim 2,
wherein R¹ and R² are bonded to each other to form a ring structure.

18. The compound according to claim 2,
wherein L¹ represents the divalent linking group represented by General Formula (3),
Rⁱ represents *-L^{S1}-aromatic hydrocarbon ring group, or
R¹ and R² are bonded to each other to represent *-L^{S2}-divalent aromatic hydrocarbon ring group-L^{S2}-*,
where, L^{S1} and L^{S2} each independently represent a single bond or a divalent linking group, and * represents a bonding position.

19. The compound according to claim 2,
wherein R⁷ and R⁸ represent hydrogen atoms.

20. The compound according to claim 2,
wherein Ar¹ represents a benzene ring group.
